(19) United States
(12) Patent Application Publication
(10) Pub. No.: US 2004/0241215 A1
(43) Pub. Date: Dec. 2, 2004

(54) Multi-dressing system for managing skin wounds

(76) Inventor: Roger D. A. Lipman, Turnhout (BE)

Correspondence Address: Heidi A. Boehlefeld, Renner, Otto, Boisselle & Sklar, LLP, Nineteenth Floor, 1621 Euclid Avenue, Cleveland, OH, 44115-2191, US

(21) Appl. No.: 10/849,640

(22) Filed: May 19, 2004

Related U.S. Application Data
(60) Provisional application No. 60/472,273, filed on May 20, 2003.

(51) Int. Cl. A61L 15/00

(52) U.S. Cl. 424/445

(57) ABSTRACT

A system for managing a skin wound, including at least one occlusive dressing; and at least one moldable composition, in which the occlusive dressing includes an occlusive layer and a fluid-absorbing pressure-sensitive adhesive material including a mixture of an adhesive material and at least one water-soluble and/or water-swellable polymer, and the moldable composition is moldable to fill body contours adjacent and/or overlapping the skin wound and when used with the occlusive dressing substantially all portions of the skin wound can be in contact with one of the moldable composition or the occlusive dressing.

This application claims priority to provisional application Serial No. 60/472,273 filed on May 20, 2003, the content of which is hereby incorporated by reference in its entirety.

### TECHNICAL FIELD

The present-invention relates to a wound management system, compositions and methods for treating acute wounds on mammalian skin surfaces. In particular, the present invention includes use of occlusive dressings for use in, e.g., skin resurfacing procedures.

### BACKGROUND

Skin resurfacing techniques include use of lasers, dermabrasion and chemical peels with alpha-hydroxy acids, phenol or trichloroacetic acid. These techniques, and particularly laser techniques, require extensive follow-up treatment to achieve the desired goal of skin resurfacing. The incidences of cosmetic surgery are increasing. It is believed that at least 300,000 such procedures are carried out each year in North America and Europe.

Despite the large numbers of such procedures, there remains a need for materials, methods and systems for treating and dressing wounds such as laser wounds. It is desirable to accelerate the healing and to restore the functional barrier property of the skin as rapidly as possible. Skin resurfacing procedures result in predictable post-operative sequelae including facial edema, wound exudate and erythema. In addition, there may be pain, pruritis, hyperpigmentation, milia formation and acne. It is desirable to address all of these consequences of wounding and aspects of healing. Thus, dressings used should absorb wound exudate sufficiently to minimize dressing changes, and should minimize incidence of wound infection, as well as reducing pain and pruritis, and contributing to a cosmetically acceptable result.

Dressings previously known solved only some of the problems that are extant in such surgical recovery situations. In addition to the foregoing, it is desirable to provide a dressing system that addresses the various stages of recovery and that addresses issues such as adherence to contoured surfaces such as the face.

Wound healing, which is a complex but orderly process, includes several established phases. The initial injury induces a first phase that includes an acute inflammatory response, characterized by production of fluid -exudate. A second phase includes regeneration of parenchymal cells, and the migration and proliferation of both parenchymal and connective tissue cells. Finally, in a third phase there is synthesis of extracellular matrix proteins, remodeling of connective tissue and parenchymal components and then collagenization and the re-acquisition of strength at the wound margin. As used herein, “parenchymal” is a non-specific term referring to a mass of surrounding tissue, including connective tissue, and cells such as fibroblasts, that lay the groundwork for tissue regeneration in skin. Thus, the healing process may be considered to have three phases, the inflammatory phase, the proliferative phase and the remodeling phase. For convenience, these phases are referred to herein as the first, second and third phases, respectively. It is noted that, in general, these three phases overlap to some degree, and thus are not wholly distinct.

A system for managing the healing of skin surface wounds, such as those resulting from skin resurfacing procedures is needed.

### SUMMARY

In one embodiment, the present invention relates to a system for managing a skin wound in an animal, including at least one occlusive dressing; and at least one moldable composition, wherein the occlusive dressing includes an occlusive layer and a fluid-absorbing pressure-sensitive adhesive material layer including a mixture of an adhesive material and at least one water-soluble and/or water-swellable polymer, wherein the moldable composition is moldable to fill body contours of the animal adjacent and/or overlapping the skin wound and when used with the occlusive dressing substantially all portions of the skin wound can be in contact with one of the moldable composition or the occlusive dressing. In one embodiment, the skin wound is a skin resurfacing wound. In one embodiment, the system further includes a moldable dressing and/or a moisturizing dressing.

In one embodiment, the invention relates to a system for managing a skin wound, including (a) at least two occlusive dressings, wherein a first of the at least two occlusive dressings exhibits a moisture absorbing capacity greater than about 3000 g/m²/24 hours, and a second of the at least two occlusive dressing exhibits a moisture absorbing capacity in a range from about 300 g/m²/24 hours to about 5000 g/m²/24 hours, and wherein each of the at least two occlusive dressings comprises an occlusive layer and a fluid-absorbing pressure-sensitive adhesive material layer comprising a mixture of an adhesive material and at least one water-soluble and/or water-swellable polymer, wherein the first occlusive dressing is applied first and is subsequently replaced by the second occlusive dressing; (b) a moldable composition moldable to fill body contours adjacent and/or overlapping the skin wound and that, when used with the at least one occlusive dressing, substantially all portions of the skin wound can be in contact with one of the moldable composition or the at least one occlusive dressing; and (c) at least one moldable and/or moisturizing dressing.

The occlusive, moldable and moisturizing dressings may include absorbing materials, pharmaceutical agents, therapeutic agents, moisturizing agents, or other agents that assist in the healing process.

In one embodiment, at least one moisturizing agent is used with one or more of the dressings. In one embodiment, the moisturizing agent is applied following use of one or more the above-described dressings.

In one embodiment, the present invention relates to a method of managing a skin wound, including applying the system disclosed herein to a patient in need thereof, as determined by a suitable health care provider.

In one embodiment, the present invention relates to a method for managing a skin wound, including applying to a patient in need thereof a system of wound dressings, the system including at least one occlusive dressing; and at least one moldable composition, wherein the occlusive dressing comprises an occlusive layer and a fluid-absorbing pressure-sensitive adhesive material layer comprising a mixture of an adhesive material and at least one water-soluble and/or water-swellable polymer, wherein the moldable composition is moldable to fill body contours of the animal adjacent and/or overlapping the skin wound and when used with the occlusive dressing substantially all portions of the skin wound can-be in contact with one of the moldable composition or the occlusive dressing, and the step of applying includes first applying the at least one moldable composition to cover portions of the skin wound that otherwise would not be contacted by the occlusive dressing, and second applying the at least one occlusive dressing to contact the skin wound and the moldable composition.

In one embodiment, the method further includes removing the occlusive dressing and applying the at least one moldable and/or moisturizing dressing to the wound.

In one embodiment, the invention relates to a wound management kit, that includes at least one occlusive dressing for initial application to the skin wound, the occlusive dressing comprising a fluid-absorbing pressure-sensitive adhesive material layer comprising a mixture of an adhesive material and at least one water-soluble and/or water-swellable polymer; and at least one moldable and/or moisturizing dressing for application to the wound after removal of the occlusive dressing.

A particular problem to be overcome in designing a dressing system for the facial area relates to the contoured nature of the surface to be dressed, particularly, for example, during the first phase of healing of laser resurfacing wounds, when exudate levels are high. The most difficult areas of all to dress are the peri-nasal and peri-oral areas. In one embodiment, the present invention resolves these difficulties by providing an occlusive dressing for the first exudative healing phase that is moldable to the contours of the face. The moldable dressing component stays where adhered under the first phase dressing, even on these difficult-to-adhere body parts such as the nose and its junction with the face. In another embodiment, the method also provides moldable and/or moisturizing dressings for later healing phases that obviate the use of creams, ointments, vegetable fats and petrolatum, and that reduce pain, provide patient comfort, and reduce or eliminate the incidence of pruritis. The compositions and methods of this invention are equally applicable to facial resurfacing wounds using chemical peel techniques, such as those resulting from the use of trichloracetic acid or phenol. The compositions and methods of this invention may also be utilized on wounds other than those resulting from application of lasers, especially on other acute wounds such as donor sites and burns. The compositions and methods of this invention may be used on skin in any area of the body, and may also be used to increase the moisture content of the skin in a controlled manner.

In one embodiment, at least one of the foregoing dressings is in the form of a mask for covering portions or all of a patient's face. Such a mask may have prefabricated openings, for example, corresponding to eyes, nasal openings and mouth. In one embodiment, the mask is split into sub-facial parts, either latitudinally, longitudinally, both latitudinally and longitudinally, or in another way. Such a mask provides another means of addressing the problems discussed above.

It will be understood by those skilled in the art that the three healing phases discussed above are not rigidly defined. For example, the second proliferative phase will in practice overlap the first inflammatory phase, and will begin before the first phase is complete. And the third remodeling phase will begin while the proliferative phase is still in progress. Further, the healing phases will vary widely with the skin type of the patient, and according to the detailed procedure being performed. Thus, in the present invention, definition of dressings for the first, second and third healing phases is made for convenience only, and no limitations are intended to be placed on the invention by so doing. Indeed, it is a further objective of the present invention to provide the plastic surgeon, the dermatologist and their nursing staff with a completely flexible wound management system; a system that is adaptable to the precise needs of the individual patient, as determined by clinical assessment at the time of dressing application or change. In practice, for example, use of the second phase dressing may well extend into the third remodeling wound healing phase, or use of the moldable dressing may extend into the proliferative healing phase, and so on, according to the judgment of the health care professional.

### BRIEF DESCRIPTION OF THE DRAWINGS

FIG. 1 is a schematic cross-sectional view of an occlusive dressing in accordance with an embodiment of the present invention.

FIG. 2 is a schematic cross-sectional view of an occlusive dressing including an adjunct absorbing layer in accordance with another embodiment of the present invention.

FIG. 3 is a schematic cross-sectional view of a moldable or moisturizing dressing in accordance with another embodiment of the present invention.

FIG. 4 includes a group of graphical representations of comparative data between treatments at post-wound intervals.

FIG. 5 includes a group of graphical representations of comparative data between treatments at post-wound intervals.

FIG. 6 is a schematic depiction of a wound treatment kit, in accordance with an embodiment of the present invention.

FIG. 7 is a schematic depiction of a dressing in the form of a mask, in accordance with an embodiment of the present invention.

It should be appreciated that for simplicity and clarity of illustration, elements shown in the Figures have not necessarily been drawn to scale. For example, the dimensions of some of the elements may be exaggerated relative to each other for clarity. Further, where considered appropriate, reference numerals have been repeated among the Figures to indicate corresponding elements.

### DETAILED DESCRIPTION

It should be appreciated that the process steps and structures described below do not form a complete process flow for manufacturing and using a complete system for managing a skin wound. The present invention can be practiced in conjunction with techniques currently used in the art, and only so much of the commonly practiced process steps are included as are necessary for an understanding of the present invention.

In one embodiment, the present invention relates to a system for managing a skin wound, including at least one occlusive dressing; and at least one moldable or moisturizing dressing, in which the at least one occlusive dressing comprises a fluid-absorbing pressure-sensitive adhesive material layer comprising a mixture of an adhesive material and at least one water-soluble and/or water-swellable polymer, and the at least one occlusive dressing is first applied to a skin resurfacing wound, and the at least one moldable and/or moisturizing dressing is applied to the wound together with the occlusive dressing or dressings.

Thus, in accordance with one embodiment of the present invention, a system for and a method of treating a wound are provided, including the sequential use of one or more occlusive dressings in the initial stages of wound healing, together with at least one of a moldable dressing and/or a moisturizing dressing. The moldable dressing is substantially similar to the moldable paste used with the occlusive dressing, but it provided in a sheet form protected by release liners. The moldable dressing may be applied either alone or together with any of the other dressings. The moisturizing dressing includes moisturizing agents for use in latter stages of healing, when the skin is substantially re-epithelialized but has not yet fully developed its barrier properties and is apt to dry out if not moisturized from an external source. In addition to these dressings, a moisturizing agent may be applied together with the dressings or in the final stages of wound healing in the absence of any dressing other than the moisturizing agent. As will be understood from the foregoing and following description of the invention, the present invention further relates to a method of managing a skin wound, including applying the above-described system to a patient in need thereof.

In one embodiment, the skin wound is a skin resurfacing wound, such as a laser resurfacing treatment wound. In one embodiment, one-or more of the fluid-absorbing pressure-sensitive adhesive dressings are applied directly to the wound.

Occlusive Dressings As described above, the occlusive dressing or dressings is applied soon after wound creation, in the initial stages of healing during which the wound may heavily exude and in subsequent stages during which the wound may continue to exude, although at a reduced rate. In order to provide dressings appropriate to these differing rates of exudation, the present invention includes two occlusive dressings having different absorbencies. The occlusive dressings are designed to be highly absorbent, while at the same time providing protection, covering and moisture retention to the wound. The occlusive dressings include a fluid-absorbing pressure-sensitive adhesive material, that provides both adhesion to the patient in the vicinity of the wound and absorbent capability for the heavy exudates that may be produced by a wound in the early stages of healing. In one embodiment, the fluid-absorbing pressure-sensitive adhesive includes a mixture of an adhesive material and at least one water-soluble and/or water-swellable polymer. The water-soluble and/or water-swellable polymer provides the fluid-absorbing capability of the pressure-sensitive adhesive in the occlusive dressing. As noted above, in addition to these functions, the occlusive dressings also provide a moisture-retention function, to avoid allowing the wound to dry out, and protective function to keep the wound clean and to help avoid further trauma to the wound area.

The fluid-absorbing pressure-sensitive adhesive material according to one embodiment of the invention includes one or more solid, physically cross-linked thermoplastic elastomer components such as styrene-olefin-styrene copolymers and a liquid rubber component that, in some embodiments, is substantially resin free. The adhesive material provides “dry tack” to adhere the adhesive to dry, i.e., not moist, skin. Dispersed within the adhesive material is a fluid-absorbing material such as an absorbent polymer. Useful as the absorbent polymer are, for example, insoluble calcium alginate and synthetic insoluble absorbents such as crystalline sodium carboxymethyl cellulose. In another embodiment, water-soluble hydrocolloids may also be used for this fluid-absorbing material.

In addition to the fluid-absorbing pressure-sensitive adhesive, in one embodiment, the occlusive dressings also include an occlusive top layer of a film material, such as a polymeric film. The film may be a continuous web or may be a non-woven fibrous web. In one embodiment, the occlusive top layer provides the occlusive function to the occlusive dressing. The occlusive top layer may provide a selected or controlled degree of moisture transmission, as described in more detail below.

In one embodiment, the fluid absorbing adhesive comprises a continuous phase including the adhesive material and, a discontinuous phase dispersed within the continuous phase and comprising the at least one water-soluble and/or water-swellable absorbent polymer.

Adhesive Material of Occlusive Dressing

The adhesive material of the fluid-absorbing pressure-sensitive adhesive of the occlusive dressings of the present invention may include a variety of pressure-sensitive adhesive materials known in the art. In one embodiment, the adhesive may include any medical grade adhesive. The medical adhesives include suitable acrylic based pressure sensitive adhesives (PSAs), suitable rubber-based pressure sensitive adhesives and suitable silicone pressure sensitive adhesives.

Useful rubber-based PSAs include those taught in U.S. Pat. No. 5,705,551 (Sasaki et al.) and in U.S. Pat. No. 4,080,348 (Korpman), the disclosures of which are hereby incorporated by reference. Examples of polymeric rubber bases include one or more of styrene-isoprene-styrene polymers, styrene-olefin-styrene polymers including styrene-ethylene/propylene-styrene polymers, polyisobutylene, styrene-butadiene-styrene polymers, polyisoprene, polybutadiene, natural rubber, silicone rubber, acrylonitrile rubber, nitrile rubber, polyurethane rubber, polyisobutylene rubber, butyl rubber, halobutyl rubber including bromobutyl rubber, butadiene-acrylonitrile rubber, polychloroprene, and styrene-butadiene rubber.

In one embodiment, a particularly useful rubber-based adhesive is that which has a thermoplastic elastomeric component and a resin component. The thermoplastic elastomeric component contains about 55-85 parts of a simple A-B block copolymer wherein the A-blocks are derived from styrene homologs and the B-blocks are derived from isoprene, and about 15-45 parts of a linear or radical A-B-A block copolymer wherein the A-blocks are derived from styrene or styrene homologs and the B-blocks are derived from conjugated dienes or lower alkenes, the A-blocks in the A-B block copolymer constituting about 10-18 percent by weight of the A-B copolymer and the total A-B and A-B-A copolymers containing about 20 percent or less styrene.

In one embodiment, the resin component comprises a. tackifier resin for the elastomeric component. In general any compatible conventional tackifier resin or mixture of such resins may be used. These include hydrocarbon resins, rosin and rosin derivatives, polyterpenes and other tackifiers. The adhesive composition contains about 20-300 parts of the resin component per one hundred parts by weight of the thermoplastic elastomeric component. One such rubber-based adhesive is commercially available from Ato Findley under the trade name HM3210.

Useful acrylic based pressure sensitive adhesives (PSAs) include those taught in U.S. Pat. No. 5,947,917 (Carte), and U.S. Pat. No. 5,164,444 (Bernard, acrylic emulsion), U.S. Pat. No.5,623,011 (Bernard, tackified acrylic emulsion). The acrylic PSA may also be a radiation curable mixture of monomers with initiators and other ingredients such as those taught in U.S. Pat. No. 5,232,958 (Ang, UV cured acrylic) and U.S. Pat. No. 5,232,958 (Mallya et al, EB cured). The disclosures of these patents as they relate to acrylic adhesives are hereby incorporated by reference.

In one embodiment, the adhesive material may include solid rubbers such as linear or radial A-B-A block copolymers or mixtures of these A-B-A block copolymers with simple A-B block copolymers. However, the proportion of A-B block copolymers, relative to the A-B-A block copolymers, should not normally exceed 85% by weight of the (total) block copolymers. In one embodiment, the proportion is in the range from about 35 to about 85% by weight of the block copolymers, and in another embodiment, the proportion is from about 55 to about 75% by weight of the block copolymers. In one embodiment, lower amounts such as 10 to 35% by weight of the block copolymers are used. These block copolymers can be based on styrene-butadiene, styrene-isoprene, and hydrogenated styrene-diene copolymers such as styrene ethylene-butylene.

Suitable styrene-diene copolymers are exemplified by a blend of linear styrene-isoprene-styrene triblock copolymer and linear styrene-isoprene diblock copolymer. Such a material is available from Kraton Polymers as KRATON® D-1161K and has a bound styrene content of about 15% and a diblock content of 17%. A second example is a blend of linear styrene-isoprene-styrene triblock copolymer and linear styrene-isoprene diblock copolymer available as KRATON® D-1117 and which has a bound styrene content of about 17% and a diblock content of 33%.

An example of a suitable hydrogenated styrene-diene copolymer is a thermoplastic elastomer comprising a blend of clear linear triblock and diblock copolymer based on styrene and ethylene-butylene with a bound styrene of 14% mass. Such a material is commercially available from Shell Chemical Company as KRATON® G-1657. Another example is KRATON® G-1652 from Shell Chemical Company, which is a thermoplastic elastomer comprised of a clear linear triblock copolymer based on styrene and ethylene-butylene, S-E/B-S, with a bound styrene content of about 30% by weight. Also suitable are polymers in which there is a combination of chemically saturated blocks and chemically unsaturated blocks. For example, a branched copolymer consisting of two polyisoprene chains attached to the rubber midblock of a styrene/ethylene-butylene/styrene triblock copolymer. Such a material is available from Shell Chemical Company as KRATON®) Research Product RP6919. This material has a styrene content of 18%, and isoprene content of 36% and an ethylene-butylene content of 46% by weight. Also, a low styrene synthetic copolymer of butadiene and styrene, commonly called SBR rubber, can be used as a solid rubber.

In one embodiment, the adhesive material includes physically cross-linked domains or areas. In another embodiment, the adhesive material is free of physically cross-linked domains or areas. As used herein, “physically cross-linked” means that the crosslinks in the polymer of which it is comprised is not of a chemical (covalent or ionic) nature but of a physical nature which means that there are areas or domains within the elastomer that have a high crystallinity, i.e., a high glass transition temperature. Physically crosslinked may also be referred to as “pseudo cross-linked”. For example, in a styrene-isoprene block copolymer, the polystyrene regions tend to associate into glassy islands or domains. As known in the art, a polymer may include either or both of physical and chemical (covalent or ionic) crosslinks. Thus, in some embodiments of the present invention, the polymers of the adhesive material may comprise either or both of physical or chemical crosslinks. In one embodiment, only physical crosslinks are present. In another embodiment, only chemical crosslinks are present.

In one embodiment, the adhesive material includes a silicone-based pressure-sensitive adhesive. Useful silicone pressure sensitive adhesives include those commercially available from Dow Corning Corp., Medical Products and those available from General Electric. Examples of silicone adhesives available from Dow Corning include those sold under the trade names BIO-PSA X7-3027, BIO-PSA X7-4919, BIO-PSA X7-2685, BIO-PSA X7-3122 and BIO-PSA X7-4502. Additional examples of silicone pressure sensitive adhesives useful in the present invention are described in U.S. Pat. Nos. 4,591,622, 4,584,355, 4,585,836 and 4,655,767.

In one embodiment, liquid rubbers may be added to the adhesive material to adjust or control the adhesive or other characteristics. Liquid rubbers useful in this embodiment of the invention include synthetic liquid isoprene rubber, depolymerized natural rubber, various functionally terminated synthetic liquid isoprene-styrene rubbers and liquid isoprene rubbers, liquid isoprene-styrene copolymer, liquid isoprene-butadiene copolymer, liquid butadiene-styrene copolymer and hydrogenated versions of these materials such as liquid ethylene-propylene-styrene. These liquid rubbers are generally compatible with the solid rubber. The liquid rubbers typically have a molecular weight of 25,000 to 50,000, a glass transition temperature of less than −50° C., and a viscosity at 38° C. of 50 to 10,000 Pas. A block copolymer of styrene and isoprene having a styrene content of about 13% and an isoprene content of about 87%, a glass transition of about −60° C., a melt viscosity of about 240 Pas at 50° C. and which is commercially available from Shell Chemical Company as LVSI101, is particularly useful in the practice of the invention. Within the adhesive material, in one embodiment, the weight ratio of solid rubber to liquid rubber is in the range from about 100:1 to about 1:2, and is varied in order to obtain the desired degree of adhesiveness and tackiness. In one embodiment, the weight ratio of solid rubber to liquid rubber is in the range from about 50:1 to about 5:1, and in another embodiment, from about 20:1 to about 10:1.

In one embodiment, the adhesive material may also include a tackifier. Tackifiers are generally hydrocarbon resins, wood resins, rosins, rosin derivatives, and the like. It is contemplated that any tackifier known by those of skill in the art to be compatible with elastomeric polymer compositions may be suitable for use with the present embodiment of the invention. One such tackifier is Wingtak 10, a synthetic polyterpene resin that is liquid at room temperature, and sold by the Goodyear Tire and Rubber Company of Akron, Ohio. Wingtak 95 is a synthetic tackifier resin also available from Goodyear that comprises predominantly a polymer derived from piperylene and isoprene. Other suitable tackifying additives may include Escorez 1310, an aliphatic hydrocarbon resin, and Escorez 2596, a C₅-C₉ (aromatic modified aliphatic) resin, both manufactured by Exxon of Irving, Tex. A variety of different tackifying additives may be used to practice the present invention.

In one embodiment, the adhesive material is free of any added tackifier.

In other embodiments, additional materials may be included in the adhesive material to modify the properties as needed for certain uses. The quantities to be added depend on the particular uses for which the dressings are to be put. Materials such as low molecular weight polyolefins, for example, polybutenes, commercially available under the tradename PARAPOL® 1300 (Exxon) or low molecular weight polyisobutylenes, commercially available under the tradenames HYVIS® 30 or INDOPOL® (BP), rubbers such as butyl rubber and high molecular weight polyisobutylene, mineral oil, and small amounts of other optional ingredients may be added. The optional low molecular weight polyisobutylene may, for example, be selected from one or more low molecular weight polyisobutylenes having a viscosity average molecular weight of from 36,000 to 70,000. Such polyisobutylenes are commercially available under the trademark VISTANEX® from Exxon Chemical as grades LMMS, LMMH and LMH, having viscosity average molecular weights of about 45,000, 53,000 and 63,000 respectively. The optional low molecular weight polyisobutylene may be present in an amount corresponding to from 0 wt. % to about 80 wt. % of the adhesive material.

Optionally, an elastomeric polymer such as butyl rubber or high molecular weight polyisobutylene may also be blended into the adhesive material. The optional butyl rubber may be used in the viscosity average molecular weight range of 200,000 to 600,000 and is exemplified by the grades Butyl 065 or Butyl 077, both available from Exxon Chemical. The optional high molecular weight polyisobutylene may be used in the viscosity average molecular weight range of 800,000 to 2,500,000 and is exemplified by the VISTANEX® MM series of products, available from Exxon Chemical, with the MM L-80 grade being a preferred grade for the optional high molecular weight polyisobutylene. The optional high molecular weight rubbers, blended as is indicated above, may be added in amounts suitable to modify various properties of the final formulation and may be from 0% to about 50% of the total weight of the adhesive material, and in one embodiment from about 0.5% to about 25% of the total weight of the adhesive material, and in one embodiment from about 5% to about 10% of the total weight of the adhesive material. The optional low molecular weight polybutenes and/or mineral oil may be added in amounts from 0% to about 20% of the weight of the adhesive material and in one embodiment from about 0.5% to about 10% of the total weight of the adhesive material, and in one embodiment from about 0.5% to about 5% of the total weight of the adhesive material.

Another optional ingredient is a polymer stabilizer. The addition of polymer stabilizers can be advantageous, to protect an unsaturated elastomer from degradation during processing. Suitable stabilizers useful in the practice of the invention include those normally indicated for use with styrenic elastomers such as organophosphites and the so-called hindered phenols, but any suitable stabilizers may be employed. An example of an organophosphite stabilizer is tris(nonylphenyl) phosphite, available as POLYGARD® HR, manufactured by Uniroyal. Particularly useful are the hindered phenols, IRGANOX® 1010 and IRGANOX® 565, manufactured by Ciba-Geigy Corporation. IRGANOX® 1010 is benzenepropanoic acid, 3,5-bis(1,1-dimethylethyl)-4-hydroxy-2,2-bis[[3-[3,5-bis(1,1-dimethylethyl)-4-hydroxyphenol]-1-oxopropoxy]methyl]-1,3-propanediyl ester. IRGANOX® 565 is 4-[[4,6-bis(octylthio)-1,3,5-triazine-2-yl]amino]-2,6-bis (1,1-dimethylethyl)-phenol. Stabilizers may be used separately or in combination, and suitable ranges are within about 0.1% to about 1.5%, and in one embodiment, from about 0.3% to about 1%, by weight based on the total formulation. When present, the stabilizers are generally added to the adhesive material, as is shown in the examples.

Water-Soluble and/or Water-Swellable Polymer of Occlusive Dressing

In one embodiment, the water-soluble and/or water-swellable polymer comprises one or more hydrophilic absorbent polymers that are soluble or that absorb and/or are swellable in water. In one embodiment, the hydrophilic absorbent polymers include one or more of alginic acid, sodium alginate, calcium alginate, cellulose-derived material, starch or a modified starch, a copolymer of a starch or a cellulosic material, a water soluble hydrocolloid, a synthetic resin, a mannan, seaweeds, and a plant mucilage. One or more such hydrophilic absorbent polymers may be present and a mixture of soluble and insoluble hydrophilic absorbent polymers can be used. Suitable swellable hydrophilic absorbent polymers include, e.g., cross-linked sodium carboxymethyl cellulose, crystalline sodium carboxymethyl cellulose, cross-linked dextran and starch-acrylonitrile graft copolymer. Others are discussed below. The swellable polymer may also be a so-called “super absorbent” material such as starch sodium polyacrylate. In one embodiment, the swellable polymer is other than a “super absorbent” material. Other hydrophilic absorbent polymers such as gluten and polymers of methyl vinyl ether and maleic acid and derivatives thereof may also be included with the water-soluble and/or water-swellable polymer.

In one embodiment, the water-soluble and/or water-swellable polymer comprises one or more hydrophilic absorbent polymers. In one embodiment, the hydrophilic absorbent polymer includes one or more of alginic acid, sodium or calcium alginate, a carboxymethyl cellulose salt (e.g., sodium, calcium, or other alkali or alkaline earth ions), a water soluble hydrocolloid, a cross-linked carboxymethyl cellulose salt, a crystalline carboxymethyl cellulose salt, a cross-linked dextrin, a starch-acrylonitrile graft copolymer, a starch polyacrylate salt, a water soluble gum, for example, pectin, guar gum or Xanthene gum, gelatin, polysaccharides and the like.

In one embodiment, the water-soluble and/or water-swellable polymer may comprise one or more water-soluble hydrocolloids, alone or blended with one or more swellable polymers. Such soluble hydrocolloids include naturally derived products such as pectin, gelatin, starches, guar gum, locust bean gum, gum arabic, gum karaya, collagen, karaya gum, alginic acid and its sodium and/or calcium salts. Also useful are the synthetic hydrocolloids such as sodium carboxymethyl cellulose, crosslinked sodium carboxymethyl cellulose, polyvinyl alcohol, polyvinyl pyrrolidone, high molecular weight polyethylene glycols and polypropylene glycols.

In one embodiment, the water-soluble and/or water-swellable polymer may comprise one or more of, e.g., starches such as flour starch, corn starch, potato starch, etc. In another embodiment, mannan such as yeast gum, manna or konjak may be included. In another embodiment, the water-soluble and/or water-swellable polymer may comprise one or more of various seaweeds such as agar-agar, sodium alginate, etc. In another embodiment, the water-soluble and/or water-swellable polymer may comprise one or more plant mucilages such as tragacanth gum, gum arabic, karaya gum, guar gum, psyllium seed gum, dammar gum, pectin etc., various proteins such as gelatin, collagen, casein, etc. In another embodiment, the water-soluble and/or water-swellable polymer may comprise one or more cellulose-derived materials such as carboxymethyl cellulose, hydroxyethyl cellulose, methyl cellulose, etc., modified starches such as soluble starch, carboxymethyl starch, dialdehyde starch, a cross-linked dextrin, etc. In another embodiment, the water-soluble and/or water-swellable polymer may comprise one or more copolymers of starch or cellulose, such as starch-acrylonitrile graft copolymer, a starch polyacrylate salt. In another embodiment, the water-soluble and/or water-swellable polymer may comprise one or more synthetic resins such as polyvinyl alcohol, sodium polyacrylate, polyethylene oxide, etc., and copolymers of starches or celluloses and acrylonitrile, acrylic acid, methacrylic acid, vinyl alcohol, vinyl chloride, etc. In some embodiment, the water-soluble and/or water-swellable polymer may comprise one or more of plant mucilages such as tragacanth gum, gum arabic, karaya gum, guar gum, psyllium seed gum, dammar gum, pectin, etc., the celluloses such as CMC (carboxymethyl cellulose), HEC (hydroxyethyl cellulose), etc., and the copolymers of starches or celluloses and acrylonitrile, acrylic acid, sulfuric acid, vinyl sulfonate, etc. The foregoing embodiments may be combined with one another, and may be combined with water swellable polymers and/or super-absorbent materials.

In one embodiment, the water swellable polymers include, for example, hydroxypropylcellulose (HPC) and polyethylene oxide (PEO). HPC is available from commercial suppliers including, for example, Aqualon, Inc., (Wilmington, DE). The useful HPC generally has an average molecular weight in the range of about 60,000 to 1,200,000. In another embodiment, the water swellable polymer includes homopolymers and copolymers of carboxymethyl cellulose, hydroxyethyl cellulose, hydroxymethyl cellulose). In another embodiment, the water swellable polymer includes a water-soluble or water-swellable polymer derived from acrylic acid or a pharmaceutically acceptable salt thereof, such as the polyacrylic acid polymers as follows: Polycarbophil (Noveon AA-1), carbomer (Carbopol 974P or 971P or 907), or a water-soluble salt of a co-polymer of methyl vinyl ether and maleic acid or anhydride (Gantrez MS-955).

The amount of hydrophilic absorbent polymer, the water-soluble and/or water-swellable polymer, may be from about 10% to about 70% of the total weight of the fluid-absorbing pressure-sensitive adhesive material, in one embodiment from about 20% to about 55% of the total weight of the fluid-absorbing pressure-sensitive adhesive material.

The hydrophilic polymer functions as the absorbent, and to help provide the “wet tack” that ensures the adhesive adheres to the skin and to mucous membranes when they are moist.

In one embodiment, the hydrophilic polymer is capable of swelling in water and transporting water.

The occlusive dressings of the present invention may be broadly divided into two groups based on the moisture absorbing capacity of the respective fluid-absorbing pressure-sensitive adhesive material of which each is comprised. In general, each of the occlusive dressings has a moisture absorbing capacity of at least 300 g/m²/24 hours. In a first embodiment, the moisture absorbing capacity is greater than about 3000 g/m²/24 hours. The occlusive dressing of the first embodiment is primarily for use as a phase 1 dressing, for heavily exuding wounds. In one embodiment, the moisture absorbing capacity is in the range from about 3000 to about 15,000 g/m²/24 hours. In another embodiment, the moisture absorbing capacity is in the range from about 6500 to about 12,000 g/m²/24 hours, and in another embodiment, from about 7500 to about 10,000 g/m²/24 hours.

In a second embodiment, the moisture absorbing capacity is in a range from about 300 g/m²/24 hours to 5000 g/m²/24 hours, and in one embodiment, from about 1000 g/m²/24 hours to about 4000 g/m²/24 hours, and in another, from about 1500 g/m²/24 hours to about 3500 g/m²/24 hours. The occlusive dressing of the second embodiment is primarily for use as a phase 2 dressing, e.g., for lightly exuding wounds, or wounds in which the initial rate of exudation has decreased, but in which some exudation continues. It will be recognized that either occlusive dressing may be substituted for the other as needed. If the occlusive dressing of the second embodiment is used in phase 1, it may be necessary to change the dressing more frequently, since its moisture absorbing capacity will be reached sooner. Just as the phases of healing are not clearly separated, the use of the first embodiment or the second embodiment of the occlusive dressing is not limited to any particular phase of the healing process. In general, however, the occlusive dressing of the first embodiment will be used first, for relatively heavily exuding wounds, and the occlusive dressing of the second embodiment will be used subsequently, when the wounds are exuding less heavily or only lightly.

The difference in water absorbency is a function both of the relative amounts of adhesive material and water-soluble and/or water-swellable polymer and of the nature of the materials included with or in the water-soluble and/or water-swellable polymer. For example, the “super-absorbent” materials may be used alone or together with any of other materials disclosed above, to increase the absorbency.

In one embodiment, the two embodiments of the occlusive dressings are sequentially applied, the first embodiment of the occlusive dressing having moisture absorbing capacity greater than about 3000 g/m²/24 hours, and the second embodiment of the occlusive dressing having moisture absorbing capacity in a range from about 300 g/m²/24 hours to about 5000 g/m²/24 hours.

The following test method may be used for determination of the dynamic absorption capacity of the dressings.

Circular samples of hydrocolloid adhesive are cut 25 mm in diameter. One side of the disc is protected by a carrier such as polyurethane or by silicone release paper. The unprotected side of the disc is placed down on a sintered glass filter funnel. The diameter of the sintered glass support should be about 10 mm greater than that of the sample. On top of the disc is placed a weight to keep the sample flat against the sintered glass; a US 5-cent coin (a “nickel”) is satisfactory.

The bottom outlet tube of the sintered glass funnel is connected by means of flexible PVC tubing to a glass tube that is clamped so as to stand in an open narrow-neck bottle containing physiological saline solution (NaCl 0.9% wt in water). The tube will reach almost to the bottom of the bottle, but must not touch any part of it. Evaporative loss of water from the saline solution can be minimized by pouring a thin layer of liquid paraffin on top of the saline in the bottle. The bottle of saline stands on a METTLER TOLEDO® electronic balance, the output of which is connected to a desktop computer. The recording of the balance is software driven such that the weight of the bottle can be regularly monitored for example on an hourly basis. By means of suitable clamps, the top of the glass sinter is adjusted to be at the level of the saline in the bottle. All air bubbles are eliminated. The balance is tared to zero, and the software is opened in run mode. As the saline is absorbed by the hydrocolloid, the weight loss in the bottle is automatically recorded for 24 hours or longer. The data can be calculated to give absorption of saline per square meter of dressing per 24 hours, and then can be plotted to give what is usually an S-shaped curve showing absorption as a function of time.

In one embodiment, the water-soluble and/or water-swellable polymer of the occlusive dressing may comprise any of the materials disclosed below with respect to the moldable dressing or the moisturizing dressing.

Other optional ingredients such as silica and optional active ingredients such as growth factors, antimicrobial compounds and wound-healing components such as collagen may also be incorporated into the adhesive compositions and/or dressings of the invention.

Occlusive Top Layer of Occlusive Dressing

In one embodiment, the occlusive top layer comprises a material that is generally impervious to fluid transmission, but which allows for some degree of moisture vapor transmission. In one embodiment, the occlusive top layer is a material that substantially prevents transmission of liquid water, but that allows transmission of water vapor. The occlusive top layer, in one embodiment, has a moisture vapor transmission rate (MVTR) of substantially zero. In another embodiment, the occlusive top layer has a moisture vapor transmission rate (MVTR) in the range of about 100 to 2000 g/m²/24 hours. In one embodiment, the MVTR ranges from about 400 to about 1000 g/m²/24 hours, and in one embodiment from about 600 to about 800 g/m²/24 hours. In one embodiment, where larger dressings are needed and treatment requires that a portion of the wound fluid be evaporated through the dressing, the MVTR may be as high as 4000 g/m²/24 hours or greater. In one embodiment, the occlusive top layer allow some degree of moisture vapor transmission, but is still occlusive overall in that it prevents a significant drying of the dressing and the underlying skin wound.

In one embodiment, the occlusive top layer is flexible yet resistant to tearing. In one embodiment, the thickness of the occlusive top layer is from about 4 micrometers (μm) to about 1500 μm (1.5 mm). In another embodiment, the thickness of the occlusive top layer is from about 15 μm to about 500 μm. In another embodiment, the thickness of the occlusive top layer is from about 20 μm to about 250 μm. In yet another embodiment, the thickness of the occlusive top layer is from about 25 μm to about 125 μm.

The occlusive top layer may be opaque or translucent. In one embodiment, it has a skin color, but other colors and patterns may be used. The occlusive top layer may be solid or porous, permeable or perforated, as adapted for the needs of the patient, as well as being a function of the composition and form of the occlusive top layer material.

In one embodiment, the occlusive top layer is substantially impervious to liquid, especially wound exudate. In yet another embodiment, the occlusive top layer is substantially impervious to bacteria. In another embodiment, the occlusive top layer is capable of absorbing liquid, especially wound exudate. In another embodiment, perforations or small apertures in the occlusive top layer facilitate a high rate moisture vapor transmission.

In one embodiment, the occlusive top layer may comprise any suitable polymeric film, plastic foam (including open celled foam), a woven fabric, knitted fabric or a non-woven fabric. In one embodiment, the fabrics may be natural or synthetic materials. In one embodiment, the occlusive top layer possesses at least some moisture vapor transmission capability, that may also be referred to as breathability.

In one embodiment, the occlusive top layer comprises a film comprised of a polymeric material including, for example, polyurethanes, polyolefins such as linear low density polyethylene, low density polyethylene and ethylene vinyl acetate, Saran materials such as vinylidene chloride copolymers of vinyl chloride, methyl acrylate, or methyl methacrylate copolymers. In one embodiment, the polymeric material is polyurethane, either as a film or as a polyurethane foam. In one embodiment, the polyurethane may be an ester or ether based polyurethane, such as MEDIFILM®. In other embodiments, materials that can be used in the occlusive top layer are styrene copolymers such as styrene-butadiene-styrene (S-B-S), styrene-isoprene-styrene (S-I-S) and styrene-ethylene/butylene-styrene (S-EB-S), methyl methacrylate copolymers, polyethylene copolymers and nitrile rubber. In other embodiments, the occlusive top layer may comprise a moisture vapor permeable film produced from synthetic polymers that are capable of being formed into continuous films by casting, extrusion or other known film-making processes.

In one embodiment, the occlusive top layer may comprise, for example, thermoplastic polyurethanes such as Dow Chemical Company's PELLETHANE®, including its 2363-80AE grade thereof; K. J. Quinn's Q-THANE®; B. F. Goodrich's ESTANE®; Mobay Chemical Company's TXIN®); and others. In other embodiments, the occlusive top layer can also comprise various polyesters, such as the copolymers of various cyclic polyesters including DuPont's HYTREL®, including its 4056 grade thereof, and General Electric's LOMOD(®, both of which are copolymers of polyether prepolymers and polybutylene terephthalate and polyisobutyl terephthalate, respectively, as well as Eastman Chemical's PCCE®. PCCE® is a copolymer of polyether and polyethylene terephthalate, such as PCCE® 9965 from Eastman Chemical Products, Inc., Other suitable flexible copolyesters are PCCE® 9966 and PCCE(® 9967 also available from Eastman. These other suitable copolyesters are characterized by the use of 1,4 cyclohexane dimethanol, 1,4 cyclohexane dicarboxylic acid, and polytetramethylene glycol ether as reactants in producing the flexible copolyester resins.

Also useful for the occlusive layer is ethylene methyl acrylate (EMA). Suitable EMAs may comprise from about 10 to about 20 wt. % methyl acrylate. EMAs are easily stretchable and conformable, and have very low, and in one embodiment, substantially zero, MVTR. In one embodiment, the occlusive layer is EMA and has an MVTR in the ranges described above. A suitable EMA is LOTRYL® 24MA005 (EMA) available from AtoChem, with a tensile strength of 2910 psi, elongation of 700%, a-melt index of 0.5, a Durometerof 84 A, a melting point of 70° C., and a Vicat Softening index of 43.

Also suitable are AtoChem LOTRYL® olefin acrylic copolymer grades 17BA01, 17BG04, 17BA04, 17BA07, 28BA175, 30BA02, 35BA40, 35BA320 (which are Ethyl Butyl Acrylate (EBA) copolymers), and 9MA02, 14MG02, 15MA03, 16MA03, 18MA02, 18MG02, 20MA08, 24MA005, 28MA07, 28MA175, 29MA03, and 35MA05 (which are Ethyl Methyl Acrylate (EMA) copolymers).

Exxon Optema® TC221 EMA is another suitable EMA.

As noted, the foregoing occlusive dressings may be used in the first and second phases of wound healing, as appropriate to the rate of exudation of the wounds under treatment.

Adjunct Moisture Absorbing Layer

In one embodiment, the occlusive dressing further comprises an adjunct moisture absorbing layer. In one embodiment, the adjunct moisture absorbing layer is a moldable pressure-sensitive hydrocolloid adhesive composition, similar to the above-described moldable dressing, except that it includes a hydrocolloid moisture absorbing material with a very high moisture-absorbing capability as the water-soluble and/or water-swellable polymer. An occlusive dressing having the adjunct layer is used when the level of exudate is expected to be very high, or when the occlusive dressing must remain in place for an extended length of time. In both situations, the adjunct layer helps to avoid leakage of wound exudate by providing extra capacity to absorb the exudate.

In one embodiment, an adjunct moisture absorbing, e.g., hydrocolloid-containing, layer is laminated between the occlusive layer and the fluid-absorbing pressure-sensitive adhesive layer. This adjunct layer may contain, in one embodiment, an absorbent material capable of absorbing 15 or more grams of physiological saline per gram of absorbent. In one embodiment, the adjunct layer comprises a “super-absorbent” polymer, as described above. Suitable super absorbent materials include the blended nonwoven rayon/“Oasis” superabsorbent fibers available from National Nonwovens, Cincinnati, Ohio. The adjunct layer may include any moldable, putty-like or paste-like hydrocolloid, but in particular may be one of the moldable hydrocolloid adhesive dressings described below.

In one embodiment, the adjunct layer comprises a foam or fibrous structure for absorbing fluids such as wound exudate. In one embodiment, the foam can absorb greater than 250%, in one embodiment at least about 500%, and in another embodiment, at least about 800%, by weight aqueous saline solution based on the dry weight of the foam. These values may be obtained using a saline absorbency test in which a dry, weighed sample is immersed for 30 minutes at 37° C. in phosphate-buffered saline containing 0.9 wt. % NaCl.

In one embodiment, the foam is substantially non-swellable. The term “substantially non-swellable” means that there is little or no increase in volume of the foam upon absorption of water or wound exudate. In one embodiment, the foam increases in volume by no greater than about 10%, and in another embodiment, by no greater than about 5%, when swell tested as above.

Suitable foams may have a wide range of thicknesses. In one embodiment, the foam is at least about 0.5 millimeter, and in another embodiment, at least about 1 millimeter thick. In one embodiment, the foam is no more than about 10-20 millimeters thick.

In one embodiment, the foam is an open-cell foam. Suitable open cell foams may have an average cell size (typically, the longest dimension of a cell, such as the diameter) of at least about 30 microns, and in one embodiment, at least about 50 microns. The cell size, in one embodiment, is no greater than about 800 microns, and in one embodiment, no greater than about 500 microns, as measured by scanning electron microscopy (SEM) or light-microscopy. In one embodiment, the foam includes a synthetic polymer that is adapted to form a conformable open cell foam that absorbs the wound exudate. Examples of suitable materials for the absorbent, substantially non-swellable foam include synthetic organic polymers including, but not limited to: polyurethanes, carboxylated butadiene-styrene rubbers, polyesters, and polyacrylates. The polymeric foams can be made of one or more types of monomers (e.g., copolymers) or mixtures (e.g., blends) of polymers. In one embodiment, the foam is a polyurethane, available under the trade designation POLYCRIL® 400 from Fulflex, Inc, Middleton, R.I. In another embodiment, the foam may be a polyester or polyether polyurethane foam that may be formed from either a linear or cross-linked polyurethane. Such foams are known in the art.

In another embodiment, the adjunct layer may comprise an absorbent foam. Such foams are known in the art, and have fluid-absorbing ability similar to the above-described foams.

In one embodiment, as described above, the fluid-absorbing pressure-sensitive adhesive material comprises a single layer, and there is no separate or adjunct absorbent layer, in addition to the single adhesive/absorbent layer of the occlusive dressing.

In one embodiment, the occlusive dressing includes an odor absorbing material, and in one embodiment, the occlusive dressings include a separate odor absorbing layer. The odor absorbing layer may include, for example, activated carbon or a molecular sieve. In one embodiment, the occlusive dressing is free of a separate odor separating layer.

In one embodiment, the occlusive dressing is free of any barrier material other than the occlusive top layer.

In one embodiment, the adhesive material and the at least one water-soluble and/or water-swellable absorbent polymer, of which the fluid-absorbing pressure-sensitive adhesive of which the adjunct moisture absorbing layer is comprised, are present as a continuous phase comprising the adhesive material and, dispersed within the continuous phase, a discontinuous phase comprising the at least one water-soluble and/or water-swellable absorbent polymer and the highly absorbent material.

Preparation of Occlusive Dressing

The adhesive compositions of the occlusive dressings of this embodiment of the invention may be prepared as in the following non-limiting example. A solid rubber, such as a styrene-olefin-styrene copolymer and, in this embodiment, a liquid rubber component, are blended together in a suitable mixer, normally a sigma blade mixer with an extruder discharge. The mixer is heated at about 170° C. A nitrogen flow of about 60 ml/sec through the mixer reduces the possibility of oxidative degradation of the rubber during processing.

About 1 part per hundred of resin (i.e., 1% phr) of a suitable stabilizer, for example, IRGANOX® 1010, may be added at this stage. Normally a small amount of the liquid rubber, for example, about 10-20% of the total to be added, is added to the whole amount of the solid rubber and the liquid rubber is allowed to blend with the soft solid rubber. When all this 10-20% of the liquid rubber has been absorbed, another portion of the liquid rubber is added, for example another 20-30% of the total to be added, and the liquid rubber is absorbed into the styrene-olefin-styrene rubber. This is continued until all the liquid rubber has been added, at which time a pourable tacky intermediate adhesive is obtained. The mixer blades are stopped, the direction of the screw is reversed, and the intermediate adhesive is removed from the mixer. It is run off into suitably release coated containers and allowed to cool. The mixer is stabilized at 90° C. and the powdery ingredients are charged to the mixer and the other optional ingredients, if present, can be added, and blended in for a period of time. After mixing at 90° C. for 20-30 minutes, the mixer temperature is raised to 105° C., and the ingredients of the adhesive material, intermediate hot melt and other low and high molecular weight rubbers if present, can then be added. If high molecular weight rubbers are used, they may need to be pre-masticated in the mixer, or pre-milled on a rubber mill. Mixing is typically continued for a further 30 minutes or so. The fully mixed mass is then removed from the mixer and then extruded or pressed to the desired thickness, and then laminated to suitable substrates.

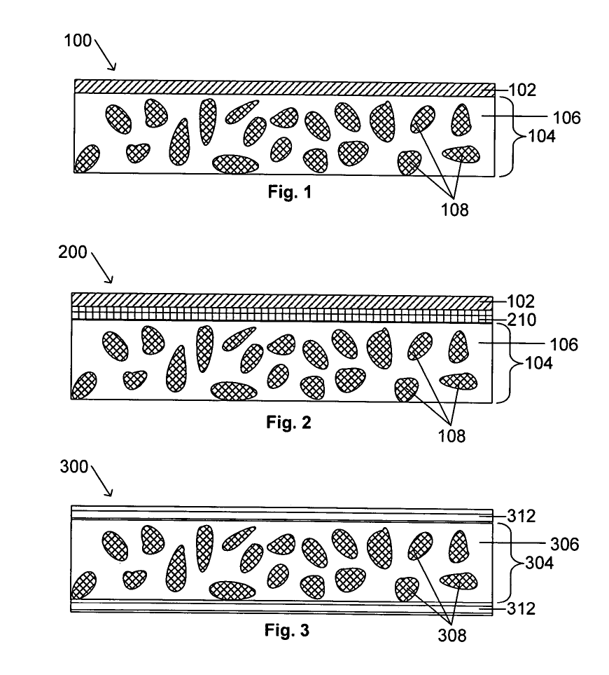

FIG. 1 is a schematic cross-sectional view of an occlusive dressing in accordance with an embodiment of the present invention. As shown in FIG. 1, an occlusive dressing 100 includes an occlusive top or upper layer 102 and a fluid-absorbing pressure-sensitive adhesive material layer 104. As described above, the fluid-absorbing pressure-sensitive adhesive material layer 104 includes an adhesive material 106 and a water-soluble and/or water-swellable polymer component 108. The adhesive material may include, for example, a thermoplastic polymer and a compatible liquid rubber, and/or any of the materials described above with respect to the adhesive material layer. The water-soluble and/or water-swellable polymer 108 may include, for example, at least one water-soluble and/or water-swellable polymer, capable of absorbing moisture or wound exudate, as described above.

FIG. 2 is a schematic cross-sectional view of an occlusive dressing in accordance with another embodiment of the present invention. As shown in FIG. 2, an occlusive dressing 200 in accordance with this embodiment includes an occlusive top or upper layer 102, a fluid-absorbing pressure-sensitive adhesive material layer 104, and an additional, or adjunct absorbing layer 210, described above. As described above, the fluid-absorbing pressure-sensitive adhesive material layer 104 includes an adhesive material 106 and a water-soluble and/or water-swellable polymer component 108. The adhesive material may include, for example, a thermoplastic polymer and a compatible liquid rubber, and/or any of the materials described above with respect to the adhesive material. The water-soluble and/or water-swellable polymer 108 may include, for example, at least one such polymer capable of absorbing moisture or wound exudate, as described above. The adjunct absorbing layer 210 may include an additional hydrocolloid absorbent material. As described above, in one embodiment, the adjunct absorbing layer 210 may include an absorbent material capable of absorbing 15 or more grams of physiological saline per gram of absorbent. This adjunct layer 210 may include any moldable, putty-like or paste-like hydrocolloid, but in particular may be one of the moldable hydrocolloid adhesive dressings described above.

Moldable Dressings

In one embodiment, the system of the present invention includes at least one moldable dressing. In one embodiment, the moldable dressing is used in the second or third phases of wound healing, and in one embodiment, in the third phase. Thus, the moldable dressing, in this embodiment, is applied subsequent to application and removal of the occlusive dressings described above. The moldable dressings described below correspond to those disclosed in GB 0110284.7 (WO 02/087646), the entire disclosure of which relating to such moldable dressings is hereby incorporated by reference. The moldable dressing, in one embodiment, comprises the moldable paste, as described below, in which the paste has been formed into and provided in a sheet form, sandwiched between two release layers.

A moldable dressing according to one embodiment of the invention comprises an adhesive material comprising a mixture of a permanently tacky pressure sensitive adhesive and a liquid rubber such as a low molecular weight polyolefin and, mixed with the adhesive material, one or more water soluble and/or water swellable absorbent polymer. Thus, the moldable dressing is similar to the occlusive dressing in that it comprises an adhesive material and one or more water soluble and/or water swellable absorbent polymer. Like the occlusive layer, in the moldable layer, the adhesive material includes a pressure-sensitive adhesive component, and the one or more water soluble and/or water swellable absorbent polymer includes a water- or moisture-absorbing component.

In one embodiment, the moldable dressing comprises a continuous phase including the adhesive material and a discontinuous phase dispersed within the continuous phase and containing at least one water-soluble and/or water-swellable absorbent polymer.

Continuous Phase of Moldable Dressing

In one embodiment, the continuous phase comprises a mixture of a permanently tacky pressure sensitive adhesive, a low molecular weight polyisobutylene and a low molecular weight liquid polybutene.

In one embodiment, the permanently tacky pressure sensitive adhesive comprises a hot melt adhesive based on a styrene-containing thermoplastic elastomer and a liquid rubber. Suitable styrene-containing thermoplastic elastomers useful in this embodiment of the invention include block copolymers based on styrene-butadiene, styrene-isoprene or styrene ethylene-butylene. In addition, a low styrene synthetic copolymer of butadiene and styrene, commonly called SBR rubber, can be used as the thermoplastic elastomer. The elastomer may comprise linear or radial A-B-A block copolymers or mixtures of these A-B-A copolymers with simple A-B block copolymers. In one embodiment, the proportion of A-B block copolymers in the mixture of A-B-A and A-B block copolymers does not exceed about 85% by weight. In one embodiment, lower percentages are used. In one such embodiment, the A-B block copolymers do not exceed 50% by weight, and in another embodiment do not exceed 40% by weight, and in yet another embodiment, do not exceed 20% by weight.

The A-B-A block copolymers are of the type that consist of A blocks derived from styrene or one of its homologues and B blocks derived from conjugated dienes, such as butadiene or isoprene, or from lower alkenes such as ethylene or butylene. The radial A-B-A polymers useful in this embodiment are of the type described for example in U.S. Pat. No.3,281,383 and conform to the general formula (A-B)ₙX, where A and B comprise blocks derived from monomers described above in connection with the A-B-A copolymers, X is an organic or inorganic connecting moiety having a functionality of at least 2, and n is equal to the functionality of X. The A-B block copolymers useful in this embodiment of the invention comprise A and B blocks derived from monomers described above in connection with the A-B-A copolymers.

Liquid rubbers useful in this embodiment of the invention include synthetic liquid isoprene rubber, depolymerised natural rubber, carboxyl terminated synthetic liquid isoprene-styrene rubber, hydroxyl terminated synthetic liquid isoprene rubber, hydrogenated liquid isoprene rubber, liquid isoprene-styrene copolymer, liquid isoprene-butadiene copolymer and liquid butadiene-styrene copolymer and combinations of two or more thereof. In one embodiment, the liquid rubber has a molecular weight in a range from about 25,000 to about 50,000. In one embodiment, the liquid rubber has a glass transition temperature of less than about −50° C., and a melt viscosity at 38° C. in the range from about 500 to about 10,000 poises. It will be appreciated that other liquid rubbers known in the art could be useful in this embodiment of the present invention.

The polyisobutylene component is exemplified by the VISTANEX® LM series of polyisobutylenes, available from Exxon Chemical Corporation, and that have Flory viscosity average molecular weights in the range from about 35,000 to about 70,000, and Brookfield viscosities at 175° C. within the range from about 20,000 to about 140,000 mpa.sec. Brookfield viscosity is determined by measuring the shearing stress on a spindle rotating at a definite, constant speed while it is immersed in the sample. Brookfield viscosity is measured in centipoises or mPa.sec. Viscosity is a function of shear rate and is defined as shear stress/shear rate.

The low molecular weight polybutene component is exemplified by the HYVIS® series of materials from BP, and by the PARAPOL® series of products from Exxon Chemical Corporation, and that have molecular weights in the range from about 1000 to about 3000, determined using test method AM-I 841-86, and kinematic viscosities at 100° C. within the range from about 180 to about 3500 cSt, as measured by test method ASTM D445.

In one embodiment, a suitable processing stabilizer may also be included in the adhesive component of the moldable dressing. Suitable stabilizers include those indicated for use with styrene-olefin-styrene block copolymer thermoplastic elastomers such as organophosphites and the so-called hindered phenols, but any suitable stabilizers may be employed. An example of an organophosphite stabilizer is tris(nonylphenyl)phosphite, available as POLYGARD® HR, manufactured by Uniroyal.

Particularly useful stabilizers are the hindered phenols, IRGANOX® 1010 and IRGANOX® 565, manufactured by Ciba. IRGANOX® 1010 is pentaerythritol tetrakis (3-(3,5-di-tert-butyl-4hydroxyphenyl) propionate. IRGANOX® 565 is 2,6-di-tert-butyl-4-(4, 6-bis(octylthio)-1,3,5-triazin-2yl-amino)phenol. Stabilizers may be used separately or in combination, and suitable ranges are within about 0.1% to about 1.5%, or in one embodiment, from about 0.3% to about 1%, by weight based on the total formulation. When present, the stabilizers are added to the adhesive material, as is shown in the examples.

Other optional ingredients such as tackifiers and plasticisers may be added to the moldable dressing, to modify tack and optimize adhesion properties.

In one embodiment, the moldable dressing is comprised of the thermoplastic elastomer and the liquid rubber, both as defined above, and is substantially resin-free. In one embodiment, the weight ratio of the thermoplastic elastomer to liquid rubber is in the range of about 1:0.5 to about 1:7, and is varied in order to obtain the desired degree of adhesiveness and tackiness.

In one embodiment, the amount of liquid rubber is also important. In one embodiment, the amount of liquid rubber corresponds to about 10 wt. % of the total formulation. In one embodiment, the maximum amount of liquid rubber is limited to about 8 wt. % of the total formulation.

In one embodiment, an amount of thermoplastic elastomer used in the moldable dressing include amounts such that moldability is not compromised. In one embodiment, the composition contains at least about 1 wt. % of the thermoplastic elastomer. However, if too much thermoplastic elastomer is present in the formulation, the compositions will be too elastic to function as a moldable material. In one embodiment, the upper limit of thermoplastic elastomer in the formulation is about 5 wt. %, in another embodiment about 3 wt. %, and in yet another embodiment, about 2 wt. %.

In one embodiment, the polyolefin component comprises low molecular weight polyisobutylene at between about 25 wt. % and about 45 wt. % of the total formulation, and in one embodiment, between about 30 wt. % and about 40 wt. % of the total formulation.

In one embodiment, the polyolefin component comprises low molecular weight polybutene at between about 5 wt. % and about 20 wt. % of the total formulation, and in one embodiment, between about 7 wt. % and about 15 wt. %.

Discontinuous Phase of Moldable Dressing

The water-soluble and/or water-swellable polymer of the moldable dressing comprises one or more hydrophilic polymers that are soluble or insoluble but swellable in water as the moisture-absorbing component. One or more swellable polymers may be present. Suitable insoluble swellable polymers include cross-linked sodium carboxymethyl cellulose, crystalline sodium carboxymethyl cellulose, cross-linked dextran and starch-acrylonitrile graft copolymer. The swellable polymer may also be a so-called “super absorbent” material such as starch sodium polyacrylate. Other hydratable polymers such as gluten and polymers of methyl vinyl ether and maleic acid and derivatives thereof may also be included in the water-soluble and/or water-swellable polymer. Suitable water soluble polymers include sodium carboxymethyl cellulose, pectin, gelatine, guar gum, locust bean gum, collagen, karaya gum and starch, particularly maize starch, and the like.

In one embodiment, the water-soluble and/or water-swellable polymer is about 60% or less of the total weight of the moldable dressing, in one embodiment, the discontinuous phase constitutes about 35% to about 55% by weight of the moldable dressing, and in another embodiment, constitutes from about 40% to about 50% by weight of the moldable dressing.

The water-soluble and/or water-swellable polymer may be comprised of any combination of soluble and/or insoluble absorbents. In one embodiment, the water-soluble and/or water-swellable polymer of the moldable dressing may comprise any of the materials disclosed herein with respect to the corresponding component of the occlusive dressing or the moisturizing dressing.

Optional fillers such as silica and pigments and optional active ingredients such as antimicrobial compounds may also be incorporated into the compositions of the moldable dressings of this embodiment of the invention. Silver sulfadiazine and benzalkonium chloride represent non-limiting examples of such antimicrobial ingredients.

The moldable dressing adhesive compositions of this embodiment of the invention may be prepared as follows. The thermoplastic elastomer and the liquid rubber component are blended together in a suitable mixer, typically a sigma blade mixer with an extruder discharge. The mixer is heated at about 170° C. A nitrogen flow of about 60 ml/sec through the mixer reduces the possibility of oxidative degradation of the rubber during processing. About 1% phr of a suitable stabilizer, for example, IRGANOX® 1010, can be added at this stage. The thermoplastic elastomer is allowed first to blend in the mixer until it coalesces. A small amount of the liquid rubber, generally about 10-20% of the total amount to be added, is added to the entire amount of the thermoplastic elastomer and the liquid rubber is allowed to blend with the softened thermoplastic elastomer. When this portion of the liquid rubber has been absorbed, another portion of the liquid rubber is added, for example about 20-30%, and the liquid rubber is absorbed into the styrene-olefin-styrene rubber. This process is continued until all the liquid rubber is added, after which a pourable tacky intermediate adhesive is obtained. The mixer blades are stopped, the direction of the screw is reversed, and the intermediate adhesive is removed from the mixer. It is poured into suitably release coated containers and allowed to cool.

The mixer is cleaned, stabilized at 90° C. and the powdery water soluble and/or water swellable polymeric ingredients are charged to the mixer together with the polyisobutylene. These ingredients are blended until uniform, approximately 10 minutes, and then the previously blended hot melt adhesive component is added and the mixer temperature is raised to 105° C. After mixing at 105° C. for 15 minutes the temperature of the mixer is dropped to about 80° C. and the low molecular weight polybutene is added. Mixing is continued typically for about another 20 minutes. The fully mixed mass is then removed from the mixer. It can then be extruded or pressed to the desired thickness, laminated to suitable substrates and die cut to shapes if desired.

The above manufacturing process has been described with respect to a two-step process in which the hot melt adhesive comprising the styrene-containing thermoplastic elastomer and the liquid rubber and the stabilizer is first manufactured and isolated and that is then subsequently used to make the compositions of this embodiment of the invention. It will be appreciated by those skilled in the art that no process limitation is implied and that the adhesives of the invention may be prepared by other methods, such as in a one-pot process, with no isolation of the intermediate hot melt adhesive.

Determination of Moldability of Moldable Dressing

Assessment of the moldability of the hydrocolloid materials is made using the test methods described in Adhesives Age, September 1997, pp 18-23. The equipment used is a probe tester manufactured by Stable Micro Systems, Godalming, Surrey, England, driven by custom designed software. Using the equipment, the energy absorbed by the moldable adhesive as the probe penetrated the adhesive is determined by a transducer. The probe is moved up and down by a rotating screw that is driven by a stepping motor. The displacement of the probe is measured through the motor rotation. For each measurement, the probe is a stainless steel ball of 25.4 mm in diameter. The compressive force is 4.5N and the test speed is 0.04 mm/sec.

Moisturizing Dressings

In one embodiment, the moisturizing dressing comprises a polyoctenamer polymer. The polyoctenamer is a viscosity modifier, that is used to control adhesive and other properties of the moisturizing dressing.

A moisturizing dressing according to this embodiment of the invention comprises an adhesive material including a mixture of a permanently tacky pressure sensitive adhesive and a trans-polyoctenamer polymer and, mixed with the adhesive material, one or more water soluble and/or water swellable absorbent polymers and, in addition to the adhesive and the polymer, a moisturizing agent. Thus, the moisturizing dressings are similar to the moldable dressing and to the occlusive dressing in that these dressings include an adhesive material including a pressure-sensitive adhesive and mixed with the adhesive material, one or more water soluble and/or water swellable absorbent polymers. However, the moisturizing dressing also includes a polyoctenamer in the adhesive material and a moisturizing agent. One example of such a moisturizing agent is glycerine. In one embodiment, the moisturizing dressing includes a high loading of moisturizing agent, for example, from about 10 wt. % to about 45 wt. %, and in one embodiment from about 20 wt. % to about 35 wt. %. It is recognized that such high loadings of moisturizing agent may cause the adhesive material to become more or less discontinuous, and in most cases, the presence of the moisturizing agent decreases the cohesive strength of the moisturizing dressing as compared to the occlusive dressings and to the moldable dressing.

In one embodiment, like the moldable dressing, the moisturizing dressing does not include a backing layer, but is provided in a sheet sandwiched between two release liners or protective layers.

In one embodiment, the moisturizing dressing comprises a continuous phase and a discontinuous phase with the continuous phase containing the pressure sensitive adhesive and the discontinuous phase containing at least one water-soluble and/or water-swellable absorbent polymer.

Adhesive Material of Moisturizing Dressing

In one embodiment, the moisturizing dressing composition of the present invention contains 0.1 to about 50% by weight, based on the adhesive material, of the polyoctenamer. In another embodiment, the moisturizing dressing composition of the present invention contains about 1 to about 35% by weight, based on the weight of the adhesive material, of the polyoctenamer. In another embodiment, the moisturizing dressing composition of the present invention contains about 5 to about 25% by weight, based on the weight of the adhesive material, of the polyoctenamer. In one embodiment, the trans-polyoctenamer is present from about 10% to about 20% by weight of the adhesive material.

The polyoctenamer component contributes to the cohesive strength of the moisturizing dressing.

A suitable polyoctenamer is available from Hüls A G of Marl, Germany, and through its distributor in the U.S., Creanova Inc. of Somerset, N.J., under the trademark VESTENAMER®. In one embodiment, the polyoctenamer is trans-polyoctenamer, which is also referred to as “trans-octenamer rubber” (TOR). Trans-polyoctenamer is a crystalline metathesis polymer from the monomer-cyclooctadiene, and is characterized by its low melting point and its ability to compound easily with other pressure sensitive adhesive components. Two grades of the VESTENAMER® trans-polyoctenamer are commercially available: “8012” identifies a material having a trans-content of approximately 80% (and a cis-content of 20%) with a melting point of approximately 54° C.; and 6213 having a trans-content of approximately 60% (cis-content of 40%) with a melting point of approximately 30° C. Both of these polymers have a double bond at every eighth carbon atom in the ring. As used herein, the term “trans-polyoctenamer” includes both of these products, and includes any polyoctenamer with a major portion of the trans-isomer. The term “polyoctenamer” refers to a polyoctenamer without regard to its trans-isomer content. Polyoctenamer compounds having other ratios of the cis- and trans-isomeric forms of the polyoctenamer can also be obtained by blending available products for use in the invention. Compounds of this class can be produced in accordance with the teachings of U.S. Pat. No. 3,804,803.

While the inventor does not wish to be bound by any particular theory of action, it is believed that the high ring content in the polymer chains serves to aid compatibility between otherwise incompatible elastomers. Also, the polymer crystallinity and the high ring content increases the amount of network formation within the pressure-sensitive adhesive material. Further, the low content of double bonds in the polymer improves the thermal and oxidative stability of the formulation, while the low viscosity of molten trans-polyoctenamer aids the processing of these materials.

In one embodiment, the moisturizing dressings contain polyoctenamer, or in one embodiment, trans-polyoctenamer, and glycerine or another skin moisturizing agent. When compounded into an adhesive, the polyoctenamer reduces the level of adhesion, and increases the cohesive strength of the adhesive. The glycerine component, at the high level used, provides excellent moisturizing action to the skin. Moreover, as the glycerine is absorbed into the skin, the loss of the plasticizing action of the glycerine is accompanied by a gradual reduction in the adhesion level. Thus, eventually, the dressing will adhere so lightly that it can be removed without any trauma whatsoever, leaving the underlying skin smooth and moisturized. The backing of the moisturizing dressing may be any suitable film, foam, nonwoven fabric, etc.

In one embodiment, the permanently tacky pressure sensitive adhesive component is tacky both at room temperature and at the skin temperature of the patient. In one embodiment, the adhesive is dermatologically acceptable, which means that after continuous contact with skin there is little adhesive residue upon removal and there is no significant reaction with the skin during the adhesion period. The adhesive strength of the adhesive material should be sufficient to adhere to the skin of the patient for the time determined by the use of the dressing of which the adhesive forms a part.

Suitable permanently tacky pressure sensitive adhesive components may be used singly or in admixture, and include natural rubber, polyisobutylene, styrene-diene block copolymers, styrene-hydrogenated diene block copolymers, butyl rubber, acrylic polymers, silicone rubber, polyurethane rubber, polyvinyl ether and other like substances. Any of the adhesive materials disclosed herein may be included in the adhesive material of the moisturizing dressing.

Other ingredients such as tackifiers, plasticisers, and polymer stabilizers may be added to the adhesive material, to modify tack and optimize adhesion properties and to protect polymers from degradation during processing.

Water-soluble and/or Water-swellable Polymer of Moisturizing Dressing

The water-soluble and/or water-swellable polymer of the moisturizing dressing comprises one or more hydrophilic polymers that are soluble or insoluble but swellable in water as the moisture-absorbing component. In one embodiment, the water-soluble and/or water-swellable polymer includes any of the hydrophilic water-absorbing materials disclosed herein with respect to the occlusive dressing or the moldable dressings. One or more swellable polymers may be present in the water-soluble and/or water-swellable polymer of the moisturizing dressing. Suitable insoluble swellable polymers include cross-linked sodium carboxymethyl cellulose, crystalline sodium carboxymethyl cellulose, cross-linked dextran and starch-acrylonitrile graft copolymer. The swellable polymer may also be a so-called “super absorbent” material such as starch sodium polyacrylate. Other hydratable polymers such as gluten and polymers of methyl vinyl ether and maleic acid and derivatives thereof may also be included in the water-soluble and/or water-swellable polymer. Suitable water soluble polymers include sodium carboxymethyl cellulose, pectin, gelatine, guar gum, locust bean gum, collagen, karaya gum and the like.

In one embodiment, the water-soluble and/or water-swellable polymer of the moisturizing dressing does not exceed 70% of the total weight of the adhesive, and in another embodiment, constitutes from about 40 to about 60% by weight of the adhesive, and in another embodiment, constitutes from about 45 to about 55% by weight of the adhesive, and may be comprised of any combination of soluble and/or insoluble absorbents.

In one embodiment, the water-soluble and/or water-swellable polymer of the moisturizing dressing may comprise any of the materials disclosed above with respect to the occlusive dressing or the moldable dressing.

Optional fillers such as silica and pigments and optional active ingredients such as epidermal growth factors and antimicrobial compounds may also be incorporated into the compositions of this embodiment of the invention. Silver sulfadiazine and benzalkonium chloride represent non-limiting examples of such antimicrobial ingredients. Also essential oils such as, for example, lavender oil or tea tree oil may be added in amounts sufficient for efficacy. Other active ingredients such as those that provide a warming or cooling sensation to the skin, for example capsaicin or menthol, may be added. Optional skin moisturizing ingredients such as urea and polyols may be incorporated into the formulations of the instant invention.

The adhesive compositions of the moisturizing dressing embodiment of the present invention may be conveniently prepared as follows. The components of the adhesive material such as polyoctenamer, solid rubber, for example a styrenic copolymer, and any liquid ingredients such as a liquid rubber or a plasticiser are blended together in a suitable mixer, typically a sigma blade or Z-blade mixer with an extruder discharge. If thermoplastic elastomers are used, the mixer will need to be heated to about 170° C. A nitrogen flow of about 60 ml/sec through the mixer reduces the possibility of oxidative degradation of the rubber during processing. About 1 phr of a suitable stabilizer can be added at this stage. After blending of the rubbers, tackifiers, plasticisers etc., the mixer is usually cooled to 90-105° C. and the powdery ingredients are charged to the mixer together with the other optional ingredients, if present, and blended in for a period of time, usually about 20-30 min. If high molecular weight rubbers are used, they may need to be pre-masticated in the mixer, or pre-milled on a rubber mill. The fully mixed mass is then removed from the mixer and extruded or pressed to the desired thickness, and then laminated to suitable substrates. The trans-polyoctenamer may be added to the contents of the mixer at the prepolymer stage, or later at the time the components of the water-soluble and/or water-swellable polymer are added. Since the trans-polyoctenamer melts at low temperatures, it is very easily incorporated into the formulations.

Other processing techniques, such as coating of the adhesive formulations from a solvent slurry, may also be employed, particularly if the desired coating weight is less than about 0.25 mm (250 μm). A general procedure for this type of processing is disclosed in U.S. Pat. No. 3,972,328, column 2, lines 44-60, and in U.S. Pat. No.4,427,737, column 2, lines 24-47. Specific process procedures are given for each of the examples below.

In one embodiment, the moldable or moisturizing layer is other than a hydrogel mask. In one embodiment, the moldable or moisturizing layer is free of a hydrogel material.

Each of the moldable dressing and the moisturizing dressing may be provided with a release liner, a backing liner, and/or a carrier material layer. In one embodiment, each of the moldable dressing and the moisturizing dressing is provided with a release liner on each major face or side. In one embodiment, during use, the moldable dressing and/or the moisturizing are used without the presence of a backing layer, carrier layer or other support layer. In one embodiment, the moldable dressing and/or the moisturizing dressing may be used with either a backing or carrier layer, which layer provides support and strength to the dressing. In one embodiment, the backing or carrier layer is in a form of a flexible, stretchable material. In one embodiment, the backing or carrier layer is an occlusive layer, i.e., the backing or carrier layer has a MVTR in the range from about zero to about 1000 g/m²/24 hours. In one embodiment, the backing or carrier layer is not an occlusive layer, i.e., the backing or carrier layer is, e.g., a non-woven fibrous material and has a MVTR in the range from about 100 g/m²/24 hours to about 2000 g/m²/24 hours. In one embodiment, the backing or carrier layer is inelastic, in that, once it is stretched to fit (for example to fit the contours of a patient's face), it does not rebound to its original shape. Thus, in this embodiment, the dressing retains the shape imparted to it upon application to the patient's body.

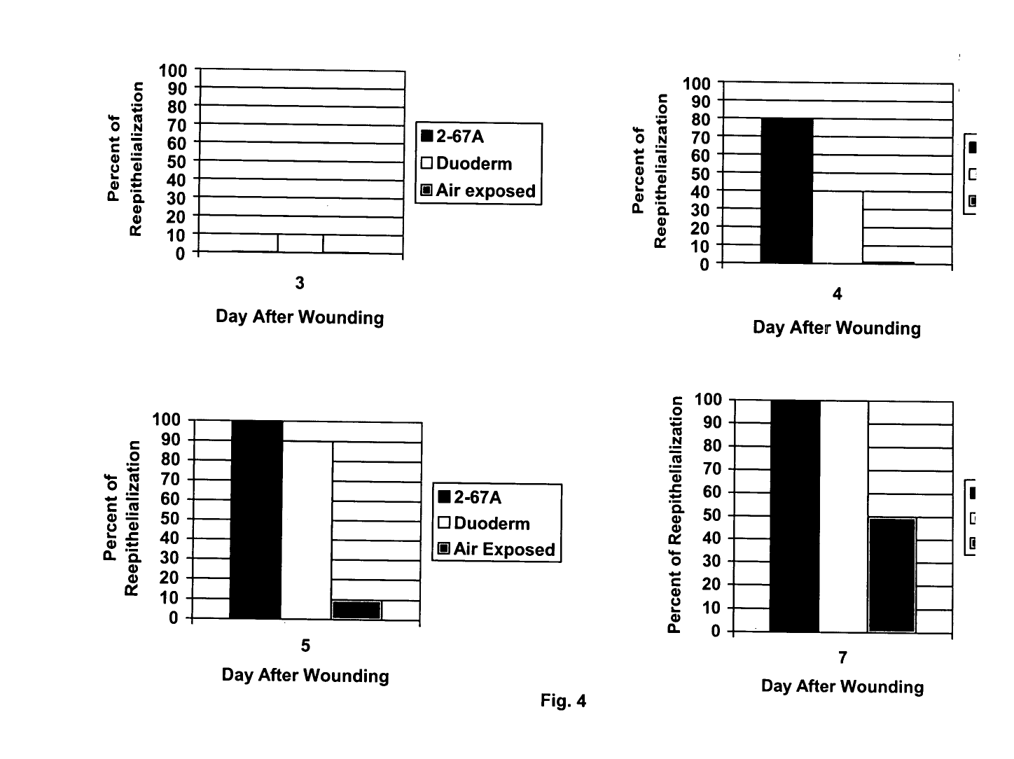

FIG. 3 is a schematic cross-sectional view of a moldable or moisturizing dressing 300 in accordance with another embodiment of the present invention. As shown in FIG. 3, a moldable or moisturizing dressing 300 in accordance with this embodiment includes top and bottom layers 312 and a fluid-absorbing pressure-sensitive adhesive material layer 304. The top and bottom layers 312 may be, independently, a release liner, a carrier layer or a backing layer. In an embodiment in which either layer 312 is a release liner, the release liner provides protection to the dressing prior to use, and is removed for application to the patient. In an embodiment in which either layer 312 is a backing layer or a carrier layer, it may be left in place, and may have any of the characteristics described above. As described above with respect to the moldable and moisturizing dressings, the fluid-absorbing pressure-sensitive adhesive material layer 304 includes a adhesive material 306 and water-soluble and/or water-swellable polymer component 308. The adhesive material may include, for example, an adhesive material including a thermoplastic polymer and a compatible liquid rubber, and/or any of the materials described above with respect to the adhesive material. The water-soluble and/or water-swellable polymer component 308 may include, for example, at least one water-soluble and/or water-swellable polymer, capable of absorbing moisture or wound exudate, as described above.

As used herein, a mask may include, a mask with openings appropriate for use on the face of a human patient, a mask with openings, separations and/or score lines appropriate for use on other body parts of a human patient, and a mask with openings, separations and/or score lines appropriate for use with non-human living beings. The latter masks may be for veterinary use, for example, in treating injured animals. Thus, a mask may include a dressing that is appropriately shaped, provided with openings, separations and/or score lines for application to various body parts of living beings as needed and as appropriate to the skin of the particular patient that is being treated.

Fluid Absorbing Adhesive Paste

In one embodiment, the present invention further relates to a fluid absorbing adhesive paste composition. In one embodiment, the paste composition is substantially the same as the material used in the moldable dressing.

In one embodiment, the fluid absorbing adhesive paste comprises a continuous adhesive phase and a discontinuous absorbing phase dispersed in the continuous phase. The continuous phase comprises a mixture of a permanently tacky pressure sensitive adhesive containing at least one styrene-containing thermoplastic elastomer and at least one compatible liquid rubber; polyisobutylene and at least one oil. In one embodiment the oil comprises mineral oil. The discontinuous phase comprises at least one water soluble and/or water swellable absorbent polymer. The adhesive paste contains about 25% to about 45% by weight, based on the total weight of the adhesive paste, of the oil.

In one embodiment, the styrene-containing thermoplastic elastomer comprises a block or radial copolymer and a liquid rubber. In one embodiment, the permanently tacky pressure-sensitive adhesive is present in a range from about 1 wt. % to about 9 wt. % of the pressure-sensitive adhesive hydrocolloid paste composition.

Suitable styrene containing thermoplastic elastomers and liquid rubber include those described above with reference to the adhesive material of the occlusive dressing and/or of the moldable dressing. In one embodiment, the weight ratio of styrene containing thermoplastic elastomer to liquid rubber is about 1:0.25 to about 1:10, and in one embodiment, from about 1:0.5 to about 1:7.

Suitable polyisobutylenes include those described above with reference to the moldable dressing. In one embodiment, the low molecular weight polyisobutylene component is present in the fluid absorbing adhesive paste composition within the range of about 25% to about 45% by weight of the paste composition.

The adhesive paste composition further includes an oil component. In one embodiment, the oil component comprises mineral oil. In one embodiment, a portion of the mineral oil may be replaced by a liquid hydrocarbon or liquid polymeric material or by a natural vegetable oil. In one embodiment, the mineral oil is exemplified by the Kaydol series of materials from Witco Chemical. Witco White Mineral Oil USP has a viscosity at 40° C. between about 60 and about 75 mm²/s, and in one embodiment, between about 63 and about 70 mm²/s, as determined by test method ASTM D-445. However, any suitable mineral oil may be used. Mineral oil may also be referred to as liquid petrolatum, mineral spirits, adepsine oil, alboline, glymol, liquid paraffin, paraffin oil or saxol, some of which may be used as trade names. Mineral oil has a boiling point in the range from about 179° C. to about 210° C.

In one embodiment, the oil component may comprise corn oil soybean oil, cottonseed oil, castor bean oil, palm oil, coconut oil, sunflower seed oil, canola oil, other known vegetable oils, animal oils such as fish oil, lard and tallow, and may further comprise synthetic oils triglycerides.

In one embodiment, the mineral oil component is present in the adhesive paste composition within the range of about 10 wt. % to about 45 wt. %, and in one embodiment, from about 15 wt. % to about 40 wt. %.

In one embodiment a portion of the mineral oil component may be replaced by a liquid hydrocarbon or a liquid polymeric component, as defined above. In one such embodiment, at least about 25 wt. % of the composition is mineral oil, and the additional liquid hydrocarbon or liquid polymer component is added thereto, up to a total limit of about 45 wt. % for the combined mineral oil and liquid hydrocarbon or liquid polymeric component.

In one embodiment, the adhesive paste composition further comprises low molecular weight polybutene. The low molecular weight polybutene components are exemplified by the HYVIS® series of materials from BP, and by the PARAPOL® series of products from Exxon Chemical Corporation.

The adhesive paste composition includes a discontinuous phase comprising at least one water-soluble and/or water-swellable polymer as a fluid absorbing component. Suitable water-soluble and/or water-swellable absorbing polymers include those described above. In one embodiment, the water-soluble and/or water-swellable polymer comprises about 25% to about 55% by weight of the total weight of the pressure-sensitive adhesive paste composition. In one embodiment, the water-soluble and/or water-swellable polymer constitutes about 30 to about 50% by weight of the pressure-sensitive adhesive paste composition, and in another embodiment, the water-soluble and/or water-swellable polymer constitutes from about 35 to about 45% by weight of the pressure-sensitive adhesive paste composition.

In one embodiment, the adhesive paste comprises a mixture of: (a) a continuous phase comprising at least one styrene-containing thermoplastic elastomer, at least one compatible liquid rubber, polyisobutylene, and at least one oil; and (b) a discontinuous phase comprising at least one water soluble and/or water swellable absorbent polymer; wherein the adhesive paste contains about 25% to about 45% by weight, based on the total weight of the adhesive paste, of the oil.

Additional details relating to the fluid absorbing adhesive paste composition may be found in copending U.S. application Ser. No. ______, entitled FLUID ABSORBING ADHESIVE PASTE (Docket No. AVERP3534USA), filed of even date herewith, the entire disclosure of which is hereby incorporated by reference.

Moisturizing Agents

In one embodiment, any of the foregoing dressings, i.e., the occlusive dressings, the moldable dressing and/or the moisturizing dressing, used in any of the healing phases, may be applied to the skin over a topically applied skin moisturizing agent, such as for example, glycerine or a formulation containing urea. In one embodiment the moisturizing agent is not used with either the moldable dressing or with the moldable paste composition. Other suitable moisturizing agents include hyaluronic acid, sodium hyaluronate, collagen, corboxymethyldextran, chitosan, squalene and lecithin. Other moisturizing agents known in the art may be used either alone or together with any of the foregoing moisturizing agents.

In one embodiment, the moisturizing agent is other than a hydrogel mask. In one embodiment, the moisturizing agent is other than a hydrogel material.

Wound Treatment Kit

In one embodiment, the invention relates to a wound management kit, which includes (a) at least one occlusive dressing for initial application to the skin wound, the occlusive dressing comprising a fluid-absorbing pressure-sensitive adhesive material comprising a mixture of an adhesive material and at least one water-soluble and/or water-swellable polymer; and (b) at least one moldable and/or moisturizing dressing for application to the wound after removal of the occlusive dressing. In one embodiment, the kit includes occlusive dressings having both disclosed absorbency capabilities. In one embodiment, the kit includes both a moldable dressing and a moisturizing dressing. In one embodiment, the kit includes (a) at least one occlusive dressing; (b) at least one moldable dressing; (c) at least one moisturizing dressing and (d) at least one fluid absorbing adhesive paste. In one embodiment, the kit includes an occlusive dressing having an adjunct high-absorbency layer. In one embodiment, the kit includes at least one moisturizing agent for topical application, for use together with or subsequent to use of any of the foregoing dressings. The kit may include numbers of each of the dressings appropriate for use in treating a single patient subsequent to a single laser skin resurfacing procedure.

In one embodiment, the kit further includes at least one mask. The mask may be prepared with any of the foregoing dressings pre-applied thereto, or the mask may be provided with no dressing applied thereto, but suitable for use with any of the foregoing dressings by combining components of the respective dressings with the mask. Any combination of the foregoing dressings, moisturizing agents, and any other materials needed for the treatment of skin is wounds such as those resulting from laser skin resurfacing procedures may be included in the kit.

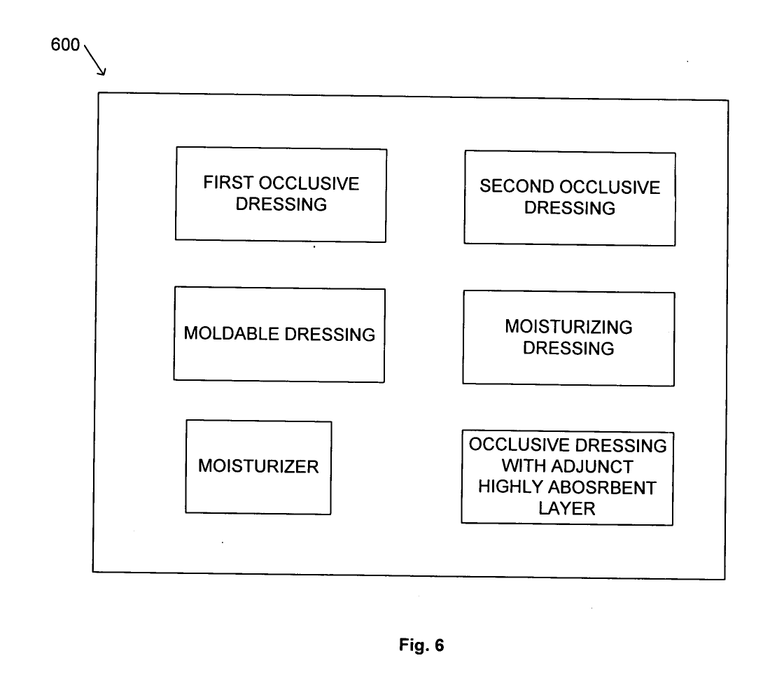

FIG. 6 is a schematic drawing of a wound treatment kit 600 in accordance with this embodiment. The kit 600 shown in FIG. 6 includes a first occlusive dressing, a second occlusive dressing, a moldable dressing, a moisturizing dressing, a moisturizer and an occlusive dressing with an adjunct highly absorbent layer. This kit includes optional elements, in accordance with one embodiment of the invention. A basic wound treatment kit includes at least one occlusive dressing for initial application to the skin wound, the occlusive dressing including a fluid-absorbing pressure-sensitive adhesive material including a mixture of an adhesive material and at least one water-soluble and/or water-swellable polymer; and at least one moldable or moisturizing dressing for application to the wound after removal of the occlusive dressing. The elements of the kit shown in FIG. 6 thus includes certain optional elements, such as the moisturizer and the occlusive dressing with an adjunct highly absorbent layer, as well as both types of occlusive dressing and both of the moldable dressing and the moisturizing dressing.

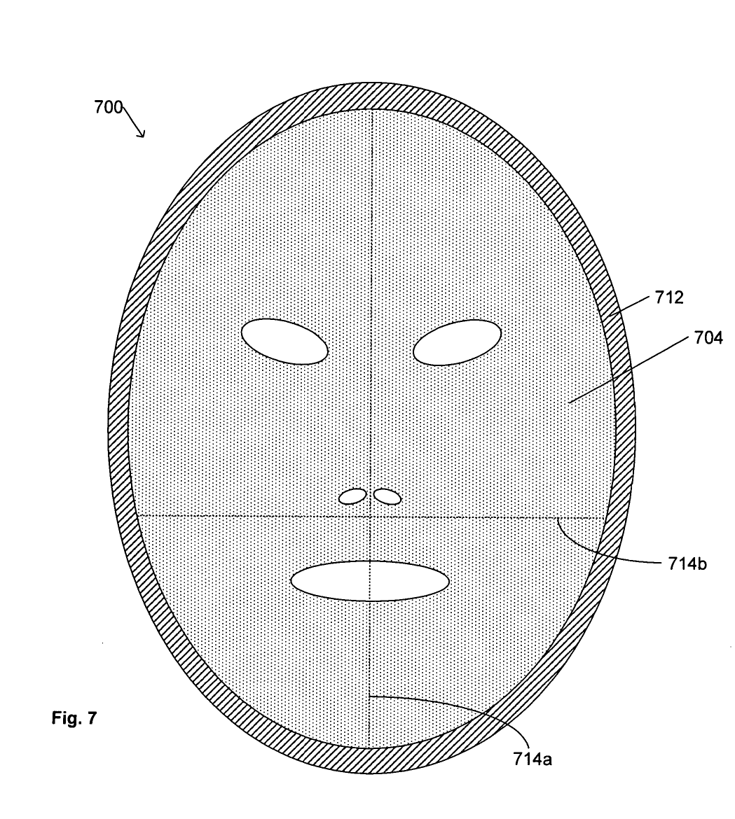

FIG. 7 is a schematic diagram of a dressing in the form of a mask, in accordance with an embodiment of the present invention. As shown in FIG. 7, in one embodiment, a dressing, such as an occlusive, moldable or moisturizing dressing as-described herein, is provided in the form of a mask 700. The mask 700 includes a dressing 704 and a backing layer 712. Although not shown, the mask may be covered by a release liner that would be removed prior to use, or may include a carrier layer, that would either be retained or be removed for prior to use. Similar layers 312 are described, for example, with respect to the embodiment shown in FIG. 3. In another embodiment, also not shown, the mask 704 may include an occlusive layer on the upper surface thereof, such as the occlusive layer 102 shown in FIGS. 1 and 2, and described with respect thereto. In another embodiment, the mask 704 may further include an adjunct highly absorbent layer, such as the layer 210 shown in FIG. 2, and described with respect thereto.

As shown in FIG. 7, the mask 704 is provided with openings appropriate for eyes, nasal openings and mouth of a patient. Such an arrangement facilitates use of the mask, by making it more comfortable, easier to apply, easier to remove, and more comprehensively covering the face than other methods, such as strip bandages.

In accordance with the present invention, the mask 704 comprises one of the dressings described above, including both the adhesive material and the water-soluble and/or water-swellable absorbent polymer component. Thus, any of the embodiments described herein may be provided in the form of the mask 704. In one embodiment of the occlusive dressings, the mask forms the occlusive layer. In one embodiment, the mask is provided for combination with any one of the foregoing dressings, the combination to be assembled at the time of use with the patient.

In accordance with another embodiment of the mask 704, the mask may be provided either in separate pieces or with scored lines to enable separation into separate pieces at the time of use. Such an embodiment is schematically shown in FIG. 7 by the dashed lines 714a and 714b. In one embodiment, the mask 704 includes a vertical separation or scored line indicated by the dashed line 714a. In one embodiment, the mask 704 includes a horizontal separation or scored line indicated by the dashed line 714b. In one embodiment, the mask 704 includes both a vertical separation or scored line indicated by the dashed line 714a, and a horizontal separation or scored line indicated by the dashed line 704b. It will be recognized that, in accordance with the invention, other separations or scored lines may be included, as needed to provide a mask 704 that is adaptable to the particular needs of physician and patient.

### EXAMPLES

The invention is further described by the following non-limiting Examples.

### Example 1

This example describes in detail the preparation of an occlusive dressing of a first embodiment, designed to handle large amounts of wound exudate. The paste adhesive material includes a hydrocolloid adhesive that utilizes an integrated network of styrene-diolefin based thermoplastic elastomer and as tackifier a liquid styrene-isoprene copolymer, with which is mixed suitable absorbent materials including the at least one water-soluble and/or water-swellable absorbent polymer.

KRATON® D-1161K, available from Kraton Polymers, is a styrene-diene copolymer comprised of a blend of linear styrene/isoprene/styrene triblock copolymer and linear styrene/isoprene diblock copolymer with a bound styrene content of about 15% and a diblock content of 17%. A processing stabilizer of the IRGANOX® 1010 described above is used to protect the thermoplastic elastomer from excessive degradation during processing. The liquid rubber is a block copolymer of styrene and isoprene having a styrene content of about 13% and an isoprene content of about 87%, a glass transition temperature of about −60° C., a melt viscosity of about 2400 poises at 50° C. LVSI-101 is used in this example.

A Z-blade mixer of 1 kg. capacity is purged with nitrogen gas and heated at 160° C. The speed of the front, faster, blade is 30 rpm. The rubber and the stabilizer are charged to the mixer at 160° C., and the mixer is started. After mixing for 5 minutes, the rubbery crumb coalesced, and 50 gm of the LVSI-101 is added with continued mixing and nitrogen purging. After a further ten minutes, the temperature is raised to 170° C. and the mixer front blade speed increased to 47 rpm. The LVSI is, at this point, completely mixed with the rubber, and a further 51 gm of LVSI is added. Ten minutes later, after blending of the second portion of the LVSI, a further 48 gm of LVSI is added, and mixed for an additional 10 minutes. In this manner, approximately 50 gm portions of the charge of LVSI are added every 10 minutes until the entire 400 gms is added. Fifteen minutes later, the intermediate adhesive is dumped from the mixer. The total time for this operation is about 90 minutes. The composition of the intermediate hot melt adhesive HMA-1 is given in the Table 1.

TABLE 1

| Formula HMA-1 | Wt % total | Amount, gm |
|---|---|---|
| LVSI-101 | 79.37 | 400 |
| KRATON ® D-1161K | 19.84 | 100 |
| IRGANOX ® 1010 | 0.79 | 4 |
| Total | 100.00 | 504 |

The mixer is cleaned and reheated to 100° C. The mixer is charged with 250.05 gm of the adhesive of HMA-1, that is heated at 100° C. with kneading. Pectin (55.55 gm), Water-Lok A-100 (97.2 gm), Water-Lok G-544 (48.6 gm), AQUASORB® A500 (48.6 gm) are added to the mixer and kneaded for 30 minutes, after which time the mixer is discharged. The hydrocolloid mixture is pressed to a sheet of 1 mm thickness between two sheets of silicone coated release paper in an upstroking platen press maintained at 90° C., and then laminated with a polyurethane film previously coated with a medical grade acrylic adhesive. The acrylic adhesive serves to anchor the film to the hydrocolloid adhesive. This adhesive coated film is available from Avery Dennison Specialty Tape Division as MED1827. The composite is cut into an A4 sheet, packaged in a peel pack and sterilized with gamma radiation at a dose of 25 KGy. The hydrocolloid adhesive had the composition shown in Table 2, and the dynamic absorption after sterilization is determined to be 5731 g/m²/24 hours.

TABLE 2

| Ingredient | Weight, gm |
|---|---|
| LVSI-101 | 198.45 |
| KRATON ® D-1161K | 49.60 |
| IRGANOX ® 1010 | 2.00 |
| Pectin USP 100 | 55.55 |
| Water-Lok A-100 | 97.20 |
| Water-Lok G-544 | 48.60 |
| AQUASORB ® A500 | 48.60 |
| Total | 500.00 |

### Example 2

This example describes in detail the preparation of an occlusive dressing of the first embodiment, designed to handle large amounts of wound exudate. In this structure, the layers form an absorbent composite comprising the hydrocolloid adhesive layer (e.g., layer 104 in FIG. 2) of HA4 of Example 4 laminated to an absorbent polyurethane foam (e.g., layer 102 in FIG. 2) prepared from HYPOL®. A layer of adhesive coated polyurethane foam (e.g., layer 102 in FIG. 2) is used for an occlusive backing layer.

To 100 parts of water in a plastic beaker are added 1.5 parts of a non-ionic surfactant PLURONIC® F-68, available from BASF Wyandotte. Then 100 parts of HYPOL® 2002, an isocyanate terminated prepolymer available from Dow Chemical Company is added, and the mixture is stirred rapidly for 20 seconds using an electric propeller mixer. The beaker contents are immediately poured into another beaker and then applied to silicone release paper held on the vacuum bed of a laboratory doctor blade drawdown apparatus. After drawdown at 2 mm thickness, the foam is allowed to cure for about 20 minutes before it is placed in a curing oven at 60° C. for several hours.

An A4 sheet of the extruded hydrocolloid adhesive of HA-4 of Example 4 below positioned between two silicone coated release papers is prepared, and circular holes of 5 mm diameter are kiss die cut in the hydrocolloid layer through the upper paper and on to the lower paper. The holes are disposed diagonally to one another and with 10 mm between them. A portion of the cured HYPOL® 2002 foam is taken measuring 18.5 cm×18.5 cm is cut and a piece of hydrocolloid measuring 20 cm×20 cm is cut. The upper hydrocolloid release paper is removed, together with the waste from the holes, and the hydrocolloid adhered to the foam with an overlap of 1.5 cm all around. A piece of non-absorbent microporous cast polyurethane foam sheet, 20 cm×20 cm, previously coated with a medical grade acrylic pressure sensitive adhesive, and available from Avery Dennison Specialty Tape Division as MED 5690U, is laminated to the top of the absorbent foam and secured to the overlapping hydrocolloid.

The dressing is packaged in a peel pouch and sterilized to 25 KGy using gamma radiation.

### Example 3

This example illustrates the preparation of adjunct moldable hydrocolloid dressing.

The hot melt adhesive, HMA-1, is prepared substantially as described in Example 1. The laboratory mixer is cleaned and AQUASORB® A500 (81.6 gm), Pectin USP100 (38.4 gm) and sodium carboxymethyl cellulose, BLANOSE® 7H4XF (96 gm) are charged to the mixer, previously heated to 90° C. After blending the powders for 2 minutes, VISTANEX® LMMS polyisobutylene (168 gm) is added, and mixing is continued for 10 minutes, following which the temperature of the mixer is raised to 105° C. The intermediate hot melt adhesive HMA-1 of Example 1 (48 gm) is added to the mixer, and blending is continued for another 10 minutes, following which the temperature in the mixer is dropped to 85° C. PARAPOL® 1300 (48 gm) is added gradually to the contents of the mixer and kneading is continued for 20 minutes after the final addition. The mixer is stopped and the hydrocolloid is removed. the hydrocolloid/adhesive mixture is extruded in a laboratory extruder between two silicone coated release papers to a web of 1 mm thickness. The hydrocolloid adhesive, HA-3, has the composition shown in Table 3:

TABLE 3
HA-3

| Ingredient | Weight, % |
|---|---|
| LVSI-101 | 7.94 |
| KRATON ® D-1161K | 1.98 |
| IRGANOX ® 1010 | 0.08 |
| VISTANEX ® LMMS | 35.00 |
| Pectin USP100 | 8.00 |
| BLANOSE ® 7H4XF | 20.00 |
| AQUASORB ® A500 | 17.00 |
| PARAPOL ® 1300 | 10.00 |
| Total | 100.00 |

### Example 4

This example describes the preparation of an occlusive dressing suitable for Phase 2 treatment, in which the exudate is not as heavy as in Examples 1 and 2. The dressing comprises an integrated hydrocolloid adhesive based on a styrene-isoprene-styrene thermoplastic elastomer and a liquid styrene-isoprene rubber, with AQUASORB®) A500 sodium carboxymethylcellulose as the absorbent filler and a polyurethane film backing.

The hydrocolloid adhesive is prepared substantially as described in Example 1 above, and as described in above with respect to the second embodiment of the occlusive dressing, using LVSI-101, a liquid rubber component that is resin free, to tackify the solid rubber component. The resulting hydrocolloid adhesive, HA-4 has the formula given in Table 4:

TABLE 4

| Example HA-4 | Wt % total | Amount, kg |
|---|---|---|
| LVSI-101 | 57.14 | 20.0 |
| KRATON ® D-1161K | 14.29 | 5.0 |
| IRGANOX ® 1010 | 0.57 | 0.2 |
| AQUASORB ® A500 | 28.00 | 9.8 |
| Total | 100.00 | 35.0 |

Mixed with the adhesive material, as the water-soluble and/or water-swellable absorbent polymer, is AQUASORB® A500, which is crystalline sodium carboxymethyl cellulose available from Aqualon, Division of Hercules Chemical Company.

The adhesive fluid-absorbing pressure-sensitive adhesive material thus prepared is extruded at 100° C. at a gauge of 0.6 mm onto release paper and is laminated in line with a polyurethane film previously coated with a medical grade acrylic adhesive to produce Dressing 4. The acrylic adhesive serves to anchor the film to the hydrocolloid adhesive. This adhesive coated film is available from Avery Dennison Specialty Tape Division as MED1827. The composite is cut into sheets, packaged in peel packs and sterilized with gamma radiation at a dose of 25 KGy.

The dynamic absorption after sterilization is determined to be 2625 gm/m²/24 hr.

### Example 5

The dressing construction, Dressing 4, described above in Example 4 is evaluated for its ability to heal partial thickness dermatome wounds on swine, an animal wound that provides a good model for the superficial wound generated by laser energy, such as is used in skin resurfacing treatments. Dressing 4 is compared in this study to a known commercial hydrocolloid dressing, DUODERM® sold by the ConvaTec Company. The wound model and the technique that is employed are described by W. H. Eaglstein and P. M. Mertz in the Journal of Investigative Dermatology, Volume 71, page 382, (1978).

Two young female specific pathogen free pigs weighing 25-30 kg are kept in house for two weeks prior to initiating the experiment. The animals are fed a basal diet ad libitum and are housed individually in animal facilities that met the American Association for Accreditation of Laboratory Animal Care regulations with controlled temperature (19-21° C.) and lights (12 h/12 h LD). Experimental animals are clipped with standard animal clippers on the day of the experiment. The skin on the back and both sides of the animal are prepared for wounding by washing with a non-antibiotic soap and tap water. Antiseptics are not used because of their potential effect on the healing process. Each animal is anaesthetized with ketamine hydrochloride (15 mg/kg), xylazine (0.2 mg/kg) and atropine (0.05 mg/kg) combination intramuscularly, followed by mask inhalation of an isoflurane and oxygen combination. Approximately 105 rectangular wounds measuring (10 mm×7 mm×0.3 mm deep) are made in the paravertebral and thoracic area with a specialized electrokeratome fitted with a 7 mm blade. The wounds are separated from one another by 15 mm of unwounded skin.

Thirty-five wounds are randomly assigned to each treatment group: Hydrocolloid Dressing 4; DUODERM® Dressing; and Air Exposed (untreated control). All wounds are made and blotted dry with sterile gauze prior to treatment. Dressings are left in place throughout the experiment. All dressings are well adhered to the skin surface. The experimental dressing appeared to absorb the wound exudate, changing the dressing color from a light brown (original color) to a cream white color in the areas that are in contact with the wounds (after the first 3 days). The experimental dressing does not leave any residue on the wounds or surrounding skin upon removal. None of the wounds in any treatment group shows signs of oedema, erythema or infection. Wounds treated with DUODERM® have some dressing residue upon removal. Neither DUODERM®) nor the experimental dressing appear to damage the wounds upon removal.

Prior to wound assessment, animals are anaesthetized as described above. Beginning on day 3 after wounding (day 0), and on each day thereafter for six days, five wounds and the surrounding normal skin from each treatment group are excised using a standard width (22 mm) electrokeratome blade set at a depth of 0.5 mm. All specimens that are not excised intact are discarded. The excised skin containing the wound site is incubated in 0.5M sodium bromide at 37° C. for 24 hours, allowing for a separation of the dermis from the epidermis. After separation, the epidermal sheets are examined macroscopically for defects.

Defects are defined as holes in the epidermal sheet or as a lack of epidermal continuum in the area of the wound. Epithelization is considered complete if no defects are present (healed); any defect in the wound area indicated that healing is incomplete. The mounted samples are retained for a permanent record. After the study is completed, the number of wounds healed (completely epithelialized) is divided by the total number of wounds sampled per day and multiplied by 100. The percentages of wounds epithelialized are plotted against days after wounding and the results are shown in FIG. 4. FIG. 4 includes a group of graphical representations of comparative data between treatments at post-wound intervals.

Day 3

Wounds treated with DUODERM® dressing are 10% completely epithelialized, while no wounds are completely epithelialized (0%) in all the other treatments.

Day 4

Wounds treated with Dressing 4 are 80% completely epithelialized while wounds treated with DUODERM® are 40% completely epithelialized. None of the wounds (0%) in the air-exposed group are completely epithelialized on this day.

Day 5

Wounds treated with Dressing 4 are completely epithelialized. Wounds treated with DUODERM® dressings are 90% completely epithelialized while only 10% of the wounds from the air-exposed group are completely epithelialized.

Day 7

All wounds treated with Dressing 4 or DUODERM® remained completely epithelialized (100%). The air exposed wounds are only 50% healed.

Both dressings increase the rate of epithelialization faster than untreated air exposed. It appears that Dressing 4 enhances the rate of epithelialization more rapidly than did DUODERM®.

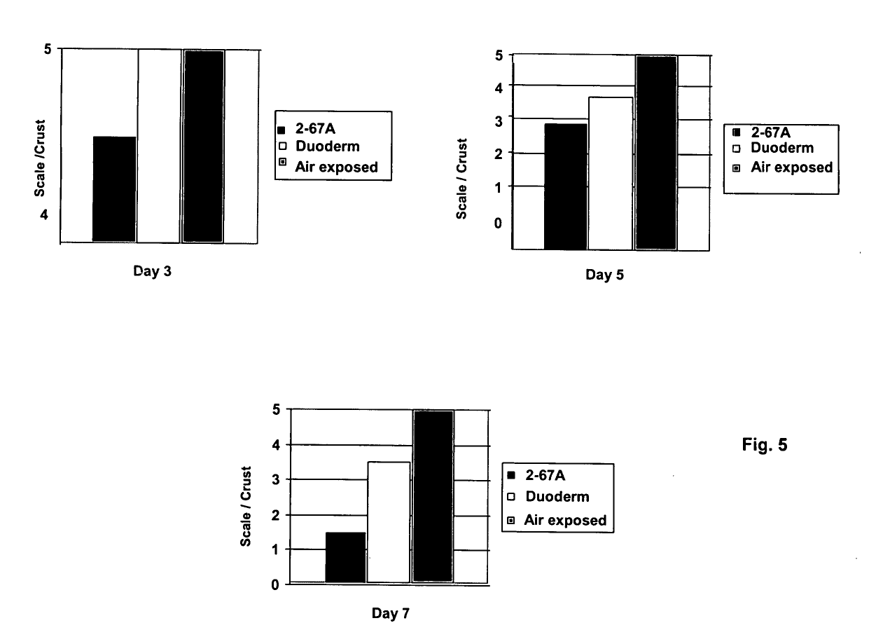

An histologic evaluation is performed as a means to observe scale/crust formation as outlined below. Excision biopsies are obtained through the center of the wounds including normal adjacent skin on both sides. Specimens are processed routinely then stained with hematoxylin and eosin. One section per block is analyzed. The specimens are evaluated via light microscopy by a dermato-pathologist who is unaware of their identity and examined for scale/crust formation. The amount of scale/crust formation are measured by the following scale: 1=absent, 2=minimal, 3=moderate, 4=marked, 5=exuberant and the results are shown in FIG. 5. FIG. 5 includes a group of graphical representations of comparative data between treatments at post-wound intervals.

Day 3

The amount of scale/crust of wounds treated with DUODERM® or left air exposed (5.0±0.0) is slightly higher than wounds treated with Dressing 4 (4.5±0.5) dressings.

Day 5

The amount of scale/crust of wounds left air exposed is higher (5.0±0.0) as compared to wounds treated with DUODERM® (3.0±2.0) or Dressing 4 (2.5±0.5).

Day 7

The amount of scale/crust of wounds left air exposed is as expected higher (5.0±0.0) as compared to wounds treated with DUODERM® (3.5±0.5), which in turn gives more scale/crust than Dressing 4 (1.5±0.5).

Both dressings reduce the amount of scale/crust compared to that resulting from untreated, air exposed wounds. However, the amount of scale/crust with Dressing 4 is less than that observed with the use of the DUODERM® dressing. By day 7, this difference is statistically significant.

### Example 6

This example illustrates the preparation of a moisturizing dressing, suitable for Phase 3 treatment. The hydrocolloid adhesive HA-4 from Example 3 is further compounded at 90° C. in a Z-blade mixer with glycerine, and trans-polyoctenamer polymer VESTENAMER® 6213. Table 5 shows the composition of the hydrocolloid adhesive dressing HA-6.

TABLE 5

| Ingredient | Weight, % |
|---|---|
| LVSI-101 | 20.78 |
| KRATON ® D-1161K | 5.19 |
| IRGANOX ® 1010 | 0.21 |
| AQUASORB ® A500 | 10.18 |
| Glycerine | 36.36 |
| VESTENAMER ® 6213 | 27.27 |
| Total | 100.00 |

The formulated adhesive is pressed at 90° C. between two sheets of silicone coated release paper, and laminated to an acrylic adhesive coated non-woven fabric commercially available from Avery Dennison Specialty Tape Division, Turnhout, Belgium as MED1817. The acrylic adhesive on the non-woven fabric serves as a tie coat to anchor the hydrocolloid adhesive to the non-woven fabric. The composite is cut to size, packaged in peel packs. It is not sterilized.

### Example 7

This example provides a clinical use study of the second and third phase dressings of the multi-dressing system, with concomitant adjuvant use of glycerine in place of CRISCO® vegetable fat. The procedure is performed as follows on a 46 year-old female following lower lid blepharoplasty and full facial resurfacing with a C0₂ laser.

Operative day 0

Patient is in surgery at approximately 13:00; is out of surgery at approximately 16:30; and is discharged from the hospital at 21:30. General anesthesia is used.

The dressing of Example 4 is applied in the Operating Room. The dressing is applied in patches and is held in place with ACE® Bandage. There is much exudate, with some leakage overnight into the ACE® bandage. VASELINE® is applied liberally peri-orbitally. Ice packs are placed on the eyes.

Post-Operative day 1

Dressing change in surgeon's office at 11:00. Example 4 dressing reapplied. Patches applied over face and retained with ACE® Bandage. Exudation generally contained, but some leakage into ACE® Bandage. VASELINE® applied liberally peri-orbitally. Ice packs used.

Post-Operative day 2

The dressing is changed in the surgeon's office at 07:30. The dressing of Example 4 is reapplied. This time the dressing is not retained with ACE® Bandage. There is considerable exudate leakage. The dressing tends to slide down the patient's face, particularly in the chin area, which is exacerbated by leakage of food and drink under the dressing onto the chin. VASELINE® is applied liberally peri-orbitally. Ice packs are placed on the eyes.

Post-Operative day 3

The dressing is left in place except at the chin area that is removed and patched with fresh Example 4 dressing. The dressing on the nose and peri-nasal area is removed and repatched with fresh Example 4 dressing. After repatching, DURAPORE® Tape is applied around the head and under the chin to prevent sliding of the dressing. VASELINE® is applied liberally peri-orbitally. Ice packs are placed on the eyes.

Post-Operative day 4

The patient removed the dressings and showered. New Example 4 dressing is applied. It is noted that exudation is essentially stopped. Crusting is observed on either side of nose, and on front of chin, under lower lip (where dressing did not adhere). After reapplying the dressing, DURAPORE® Tape (3M Co.) is placed around the head and under the chin. The DURAPORE® adhered well to the backing film of the Example 4 dressing. VASELINE® is applied liberally peri-orbitally. Ice packs are placed on the eyes.

Post-Operative day 5

The Example 4 dressing is dislodged on left side of face (1 ″ in front of left ear) and the uncovered skin area is crusted. The dressing is re-adhered, with tape used for attaching adjacent pieces of the Example 4 dressing to one another. The re-adhered portion of the dressing whitens overnight, indicating absorption of exudate. Inspection of this area shows that the crust has softened under the dressing. VASELINE® is applied liberally peri-orbitally. Ice packs are place on the eyes.

Post-Operative day 6

The Example 4 dressing is completely removed after showering. The DURAPORE®) Tape comes off hair easily when the hair is wet. The dressing is quite easy to remove. The patient feels no pain on removal, only minor discomfort, and no trauma to fragile skin, nor to areas of crust. There is crust around perioral area and on each side of the nose. CRISCO® vegetable fat is applied liberally to the whole of the laser-treated facial area. VASELINE® is applied liberally peri-orbitally. Ice packs are placed on the eyes.

The patient complains of incipient herpetic lesions on the lips, notwithstanding the use of 2×500 mg Valacyclovir per day from three days prior to surgery until today. Topical Acyclovir 5% ointment is started and is applied to lips 5 times per day. Famciclovir 250 mg/day for 5 days is also instituted. No open lesions occur.

Post-Operative day 8

CRISCO® is wiped away and the moisturizing dressing of Example 6 is applied during the day only to the right side of the patient's face. At night, all of the patient's face is covered with the Example 6 moisturizing dressing, except the forehead, which has application of Example 4 dressing. Patient notes that application of moisturizing dressing makes her more confident about sleeping lying prone at night. If CRISCO® gets on bedding, the patient must sleep upright.

Post-Operative day 9

In the evening, the Example 4 dressing is removed from the forehead and replaced with moisturizing dressing of Example 6. After removal of the Example 4 dressing, the liquefied crust (yellow, no odor) is wiped off with glycerine soaked sterile gauze, and more glycerine is applied to forehead under the moisturizing dressing. All through day 9, any exposed skin is liberally moistened with 100% glycerine. No further CRISCO® is used in this study.

Post-Operative day 10

The moisturizing dressing is removed at 09:30 from the forehead. With concomitant application of water from a pump spray bottle the dressing comes off with essentially zero peel force and zero trauma. Without the water spray, the dressing is a little more tenacious, but is still removable without pain and without apparent mechanical skin damage.

Moisturizing dressing on the left side of the patient's face is removed very easily at 10:45 after showering. The rest of the moisturizing dressing is removed at 12:30. A non-comedogenic skin moisturizer, such as an oil-free skin moisturizer is applied during the day. Glycerine is applied over the moisturizer morning and evening when at home.

Post-Operative day 11

The oil-free moisturizer is applied to the whole face in the morning after showering. A little glycerine is applied over the moisturizer all day. No dressing is used during this day.

Post-Operative day 13

Glycerine is applied to the skin with hydrocortisone cream because of itching. The patient's face is covered with the moisturizing dressing of Example 6.

Post-Operative day 14

The dressing from day 13 is removed and is replaced with same Example 6 moisturizing dressing with hydrocortisone cream 0.5 wt. % and glycerine is applied to the skin under the dressing to minimize itching.

Conclusion

This pilot study demonstrates the comfortable, pain reducing action of the second and third healing phase dressings, in combination with the beneficial use of glycerine as adjunct moisturizer. The second phase dressing, used in this trial during the first and second healing phases, essentially eliminates post-operative pain in this patient. The third phase moisturizing dressing also dramatically reduces itching. Further, when the skin does itch under the moisturizing dressing, patient can lightly scratch the dressing surface to get relief, without damaging the extremely sensitive skin. (Scratching of unprotected skin for example during the night may cause extensive bleeding and trauma.)

### Example 8

This Example illustrates the use of a second phase dressing of the invention, together with use of glycerine USP, to achieve a successful healing outcome in a combined open and closed treatment regimen.

A 41 year-old woman who had a full face laser resurfacing procedure using a CO₂ laser presented on day 3 post-operatively having not complied with post-operative wound management directions. A commercially available adhesive coated foam dressing had been applied to the face post-operatively. Because the patient found the dressing uncomfortable, she had removed the dressing immediately on reaching home and had stayed in bed for three days without any open or closed wound management. On presentation, her face is thickly crusted and she is in considerable pain and discomfort.

The phase 2 dressing described in Example 4 is immediately applied, and held in place with an ACE® bandage. The patient found this dressing much more comfortable, and accepted its presence. It is left in place for 2 days whereupon, after removal, it is found that the crusting had softened such that the face could be wiped and cleansed with saline soaked gauze, and most of the remaining crust came away easily by gentle removal with forceps. The patient confirmed the comfort of the Example 4 dressing and the absence of pain. A further Example 4 dressing is applied and again left in place for two days. On day 7 post operatively, the dressing is again removed, the skin is gently cleansed 10 with saline soaked gauze, and glycerine USP is applied liberally to the skin surface. The glycerine is covered with a third Example 4 dressing that is left in place for a further 48 hours. It may be necessary gently to press the dressing to get it to adhere on to the glycerine-coated skin. After a minute or two, excellent adhesion of the dressing is achieved. On day 9, the dressing is easily peeled off, and the skin had healed to a point where the patient could apply moisturizing lotions, and healing then proceeded normally to a successful outcome.

While the invention has been explained in relation to its preferred embodiments, it is to be understood that various modifications thereof will become apparent to those skilled in the art upon reading the specification. Therefore, it is to be understood that the invention disclosed herein is intended to cover such modifications as fall within the scope of the appended claims.

1. A system for managing a skin wound in an animal, comprising:
at least one occlusive dressing; and
at least one moldable composition,
wherein the occlusive dressing comprises an occlusive layer and a fluid-absorbing pressure-sensitive adhesive material layer, the adhesive material layer comprising a mixture of an adhesive material and at least one water-soluble and/or water-swellable polymer,
wherein the moldable composition is moldable to fill body contours of the animal adjacent and/or overlapping the skin wound and when used with the occlusive dressing substantially all portions of the skin wound can be in contact with one of the moldable composition or the occlusive dressing.

2. The system of claim 1, further comprising at least one moisturizing dressing for application to the skin wound after removal of the occlusive dressing.

3. The system of claim 1, wherein the at least one occlusive dressing has a moisture absorbing capacity of at least 300 g/m²/24 hours.

4. The system of claim 1, wherein at least one said occlusive dressing has a moisture absorbing capacity in a range from about 300 g/m²/24 hours to about 5000 g/m²/24 hours.

5. The system of claim 1, wherein at least one said occlusive dressing has a moisture absorbing capacity greater than 3000 g/m²/24 hours.

6. The system of claim 1, wherein two occlusive dressings are sequentially applied, a first occlusive dressing having moisture absorbing capacity greater than 3000 g/m²/24 hours, and a second occlusive dressing having moisture absorbing capacity in a range from about 300 g/m²/24 hours to about 5000 g/m²/24 hours.

7. The system of claim 2, wherein the moisturizing dressing comprises a mixture of an adhesive material and at least one water-soluble and/or water-swellable polymer.

8. The system of claim 2, wherein the at least one moisturizing dressing comprises a trans-polyoctenamer polymer.

9. The system of claim 2, further comprising at least one moisturizing agent applied with the moisturizing dressing.

10. The system of claim 1, wherein the adhesive material comprises a thermoplastic polymer and a compatible liquid rubber.

11. The system of claim 10, wherein the thermoplastic polymer comprises a block copolymer.

12. The system of claim 10, wherein the thermoplastic polymer is either a physically cross-linked solid rubber or a styrene-containing thermoplastic elastomer.

13. The system of claim 12, wherein the polymer is a styrene-containing thermoplastic elastomer and further comprises one or more low molecular weight polyolefins.

14. The system of claim 12, wherein the polymer is a physically cross-linked solid rubber comprising at least one styrenic copolymer.

15. The system of claim 10, wherein the liquid rubber comprises at least one of a liquid isoprene polymer, a liquid isoprene-styrene copolymer, and a liquid isoprene-butadiene copolymer.

16. The system of claim 1, wherein the at least one water-soluble and/or water-swellable polymer comprises at least one hydrophilic absorbent polymer.

17. The system of claim 16, wherein the hydrophilic absorbent polymer comprises one or more of alginic acid, sodium alginate, calcium alginate, cellulose-derived material, starch or a modified starch, a copolymer of a starch or a cellulosic material, a water soluble hydrocolloid, a synthetic resin, a mannan, seaweeds, a plant mucilage.

18. The system of claim 1, wherein the skin wound is a skin resurfacing wound.

19. The system of claim 1, further comprising an adjunct absorbing layer comprising a highly absorbent polymeric material, wherein the adjunct absorbing layer is between the occlusive layer and the fluid-absorbing pressure-sensitive adhesive material layer.

20. The system of claim 19, wherein the adjunct absorbing layer comprises an absorbent material capable of absorbing about 15 or more grams of physiological saline per gram of absorbent.

21. The system of claim 1, wherein at least one of the dressings is provided in the form of a mask.

22. The system of claim 1 further comprising at least one fluid absorbing adhesive paste.

23. The system of claim 22 wherein the adhesive paste comprises
a continuous phase comprising at least one styrene-containing thermoplastic elastomer, at least one compatible liquid rubber, polyisobutylene, and at least one oil; and
a discontinuous phase comprising at least one water soluble and/or water swellable absorbent polymer;
wherein the adhesive paste contains about 25% to about 45% by weight, based on the total weight of the adhesive paste, of the oil.

24. A method of managing a skin wound, comprising applying the system of claim 1 to a patient in need thereof.

25. A system for managing a skin wound, comprising:
(a) at least two occlusive dressings,
wherein a first of the at least two occlusive dressings exhibits a moisture absorbing capacity greater than about 3000 g/m²/24 hours, and a second of the at least two occlusive dressing exhibits a moisture absorbing capacity in a range from about 300 g/m²/24 hours to about 5000 g/m²/24 hours, and
wherein each of the at least two occlusive dressings comprises an occlusive layer and a fluid-absorbing pressure-sensitive adhesive material layer comprising a mixture of an adhesive material and at least one water-soluble and/or water-swellable polymer, wherein the first occlusive dressing is applied first and is subsequently replaced by the second occlusive dressing;
(b) a moldable composition moldable to fill body contours adjacent and/or overlapping the skin wound and that, when used with the at least one occlusive dressing, substantially all portions of the skin wound can be in contact with one of the moldable composition or the at least one occlusive dressing; and
(c) at least one moisturizing dressing.

26. The system of claim 25, wherein the first occlusive dressing is first applied to a skin wound, and the second occlusive dressing is subsequently applied to the wound, and thereafter the at least one moisturizing dressing is applied to the wound.

27. The system of claim 25, wherein the moisturizing dressing comprises a mixture of an adhesive material and at least one water-soluble and/or water-swellable polymer.

28. The system of claim 25, wherein the at least one moisturizing dressing comprises a trans-polyoctenamer polymer.

29. The system of claim 25, further comprising at least one moisturizing agent applied with the moisturizing dressing.

30. The system of claim 25, wherein the adhesive material layer comprises a thermoplastic polymer and a compatible liquid rubber.

31. The system of claim 30, wherein the thermoplastic polymer comprises a block copolymer.

32. The system of claim 30 wherein the thermoplastic polymer is either a physically cross-linked solid rubber or a styrene-containing thermoplastic elastomer.

33. The system of claim 32, wherein the polymer is a styrene-containing thermoplastic elastomer and further comprises a low molecular weight polyolefin.

34. The system of claim 32, wherein the thermoplastic polymer is a physically cross-linked solid rubber comprising at least one styrenic copolymer.

35. The system of claim 30, wherein the liquid rubber comprises at least one of a liquid isoprene polymer, a liquid isoprene-styrene copolymer, and a liquid isoprene-butadiene copolymer.

36. The system of claim 25, wherein the at least one water-soluble and/or water-swellable polymer comprises at least one hydrophilic absorbent polymer.

37. The system of claim 36, wherein the hydrophilic absorbent polymer comprises one or more of alginic acid, sodium alginate, calcium alginate, cellulose-derived material, starch or a modified starch, a copolymer of a starch or a cellulosic material, a water soluble hydrocolloid, a synthetic resin, a mannan, seaweeds, a plant mucilage.

38. The system of claim 25, wherein the skin wound is a skin resurfacing wound.

39. The system of claim 25, further comprising an adjunct absorbing layer comprising a highly absorbent polymeric material, wherein the adjunct absorbing layer is between the occlusive layer and the fluid-absorbing pressure-sensitive adhesive material layer.

40. The system of claim 39, wherein the adjunct absorbing layer comprises an absorbent material capable of absorbing about 15 or more grams of physiological saline per gram of absorbent.

41. The system of claim 36, wherein at least one of the dressings is provided in the form of a mask.

42. The system of claim 25 further comprising a fluid absorbing adhesive paste.

43. The system of claim 42 wherein the paste comprises
a continuous phase comprising at least one styrene-containing thermoplastic elastomer, at least one compatible liquid rubber, polyisobutylene, and at least one oil; and
a discontinuous phase comprising at least one water soluble and/or water swellable absorbent polymer;
wherein the adhesive paste contains about 25% to about 45% by weight, based on the total weight of the adhesive paste, of the oil.

44. A method of managing a skin wound, comprising applying the system of claim 25 to a patient in need thereof.

45. A method for managing a skin wound, comprising:
applying to a patient in need thereof a system of wound dressings, the system comprising:
at least one occlusive dressing; and
at least one moldable composition,
wherein the occlusive dressing comprises an occlusive layer and a fluid-absorbing pressure-sensitive adhesive material layer comprising a mixture of an adhesive material and at least one water-soluble and/or water-swellable polymer,
wherein the applying includes the moldable composition is moldable to fill body contours of the animal adjacent and/or overlapping the skin wound and when used with the occlusive dressing substantially all portions of the skin wound can be in contact with one of the moldable composition or the occlusive dressing, and
the step of applying includes first applying the at least one moldable composition to cover portions of the skin wound that otherwise would not be contacted by the occlusive dressing, and second applying the at least one occlusive dressing to contact the skin wound and the moldable composition.

46. The method of claim 45, further comprising removing the occlusive dressing and, after the occlusive dressing is removed, applying at least one moisturizing dressing to the skin wound.

47. The system of claim 46, wherein the moisturizing dressing comprises a mixture of a an adhesive material and at least one water-soluble and/or water-swellable polymer.

48. The method of claim 45, wherein the at least one occlusive dressing has a moisture absorbing capacity of at least 300 g/m²/24 hours.

49. The method of claim 45, wherein at least one said occlusive dressing has a moisture absorbing capacity in a range from about 300 g/m²/24 hours to about 5000 g/m²/24 hours.

50. The method of claim 45, wherein at least one said occlusive dressing has a moisture absorbing capacity greater than 3000 g/m²/24 hours.

51. The method of claim 45, wherein the step of applying comprises first applying the moldable composition to portions of the skin wound that otherwise would not be contacted by the occlusive dressing, and thereafter applying sequentially two occlusive dressings, comprising first applying a first occlusive dressing having moisture absorbing capacity greater than about 3000 g/m²/24 hours, removing the first occlusive dressing, and thereafter applying a second occlusive dressing having moisture absorbing capacity in a range from about 300 g/m²/24 hours to about 5000 g/m²/24 hours.

52. The method of claim 45, further comprising applying at least one moisturizing agent with the moisturizing dressing.

53. The method of claim 52, wherein at least one said moisturizing agent is topically applied.

54. The method of claim 45, wherein the skin wound is a skin resurfacing wound.

55. The method of claim 45, wherein the at least one occlusive dressing further comprises an adjunct absorbing layer comprising a highly absorbent polymeric material, wherein the adjunct absorbing layer is between the occlusive layer and the fluid-absorbing pressure-sensitive adhesive material layer.

56. The method of claim 55, wherein the adjunct absorbing layer comprises an absorbent material capable of absorbing about 15 or more grams of physiological saline per gram of absorbent.

57. The method of claim 45, wherein the at least one water-soluble and/or water-swellable polymer comprises at least one hydrophilic absorbent polymer.

58. The method of claim 57, wherein the hydrophilic absorbent polymer comprises one or more of alginic acid, sodium alginate, calcium alginate, cellulose-derived material, starch or a modified starch, a copolymer of a starch or a cellulosic material, a water soluble hydrocolloid, a synthetic resin, a mannan, seaweeds, a plant mucilage.

59. The method of claim 45, wherein at least one of the dressings is provided in the form of a mask.

60. A wound treatment kit, comprising: managing a skin wound, comprising:
at least one occlusive dressing, the occlusive dressing comprising a fluid-absorbing pressure-sensitive adhesive material comprising a mixture of an adhesive material and at least one water-soluble and/or water-swellable polymer;
at least one moldable composition; and
at least one moisturizing dressing.

61. The kit of claim 60, wherein at least one of the occlusive dressings is provided in the form of a mask.

62. The kit of claim 60, wherein at least one of the moisturizing dressings is provided in the form of a mask.

63. The kit of claim 60, further comprising a mask for use with at least one of the occlusive and/or moisturizing dressings.

64. The kit of claim 60, the at least one occlusive dressing comprising two occlusive dressings, a first occlusive dressing having moisture absorbing capacity greater than about 3000 g/m²/24 hours, and a second occlusive dressing having moisture absorbing capacity in a range from about 300 g/m²/24 hours to about 5000 g/m²/24 hours.
